(19) United States
(12) Patent Application Publication
(10) Pub. No.: US 2026/0317769 A1
(43) Pub. Date: Oct. 15, 2026

(54) APPLICATION OF A MICRORNA MOLECULE IN THE PREPARATION OF DRUGS FOR TREATING CEREBRAL ISCHEMIA

(71) Applicant: Zhejiang Chinese Medical University, Zhejiang (CN)

(72) Inventors: Huifen Zhou, Zhejiang (CN); Haitong Wan, Zhejiang (CN); Jiehong Yang, Zhejiang (CN); Yan Guo, Zhejiang (CN)

(73) Assignee: Zhejiang Chinese Medical University, Zhejiang (CN)

(21) Appl. No.: 19/218,350

(22) Filed: May 25, 2025

(30) Foreign Application Priority Data
Apr. 9, 2025 (CN) 202510436509.9

(51) Int. Cl. C12N 15/113; A61P 9/10

(52) U.S. Cl. CPC C12N 15/1137; A61P 9/10; C12N 2310/141; C12Y 302/01023

(57) ABSTRACT

The present invention discloses the use of the application of a microRNA molecule in the preparation of drugs for treating cerebral ischemia. By downregulating the expression of Glb1, it can inhibit the metabolism of GM1 in MCAO/R model rats and the metabolism of ganglioside GM1 in microglia after OGD/R. The study identified miR-6340, an upstream miRNA of the Glb1 protein-coding gene, and demonstrated that the miR-6340/Glb1 axis promotes microglial autophagy through GM1 metabolism. This application provides a novel perspective on the neuroprotective role of GM1 by reducing autophagy in OGD/R-induced injury.

### CROSS-REFERENCE TO RELATED APPLICATION

This application claims the priority benefit of China application no. 202510436509.9, filed on Apr. 9, 2025. The entirety of each of the above-mentioned patent applications is hereby incorporated by reference herein and made a part of this specification.

### REFERENCE TO A SEQUENCE LISTING

The instant application contains a Sequencing Listing which has been submitted electronically in XML format and is hereby incorporated by reference in its entirety. Said XML copy, created on May 21, 2025, is named 155967_SEQUENCELISTING and is 35,586 bytes in size.

### TECHNICAL FIELD

The invention belongs to the technical field of medicine, in particular to the application of a microRNA molecule in the preparation of drugs for treating cerebral ischemia.

### BACKGROUND OF RELATED ART

Stroke is a common and refractory emergency. It is a group of diseases caused by the sudden rupture of blood vessels in the brain or the blockage of blood vessels that prevents blood from flowing into the brain. Stroke is the first common cause of human death, and it is also the main cause of disability, seriously endangering the quality of life and health of our people. The per capita hospitalization cost of stroke patients increased by 31.4 percent over 2010, bringing a heavy economic burden to the country and society. Stroke includes ischemic stroke and hemorrhagic stroke, of which ischemic stroke, also known as ischemic stroke, accounts for 69.6% of strokes.

Recombinant tissue plasminogen activator (rt-PA) is the most commonly used intravenous thrombolytic agent for acute ischemic stroke. It activates plasminogen, converting it into plasminase, which can degrade fibrin in blood clots, thus dissolving blood clots and restoring cerebral blood perfusion. However, the narrow therapeutic window and side effects limit its clinical application, such as symptomatic intracranial hemorrhage that may occur after thrombolysis can lead to patients' condition aggravation and even life-threatening. According to statistics, the incidence of symptomatic intracranial hemorrhage after thrombolytic therapy with rt-PA is about 6%.

Aspirin and clopidogrel are commonly used antiplatelet drugs, the main problem is the risk of bleeding, long-term use may lead to gastrointestinal bleeding, especially aspirin has a direct stimulating effect on the gastric mucosa, may cause gastric ulcer and stomach bleeding.

Edaravone is a free radical scavenger, which can reduce the damage of nerve cells caused by free radicals during cerebral ischemia-reperfusion injury. However, in the process of use, it may cause kidney damage in elderly patients and patients with renal insufficiency, and its clinical efficacy is not very significant in some large-scale clinical trials.

Butylphthalein is a commonly used drug to improve cerebral circulation, which can increase cerebral blood flow and improve microcirculation in patients with acute ischemic stroke. However, the best time window for its use is within 48 hours after the onset of the disease, and the narrow treatment window also limits its clinical application, and some patients may have mild elevated aminotransferase, nausea, abdominal discomfort and other adverse reactions.

In addition to the above drug treatment methods, there are endovascular interventional therapy and angioplasty and other treatment methods, although it can quickly open the occlusive blood vessels to improve cerebral blood perfusion, but its surgical operation is more difficult, there are operational risks, in addition, postoperative carotid restenosis is also a relatively common problem.

### SUMMARY OF THE INVENTION

Aiming at the problems existing in the prior art, the design of the invention aims to provide the application of a microRNA molecule in the preparation of drugs for treating cerebral ischemia, which is specifically realized through the following technical schemes:

The first part of the invention provides the application of miR-6340 in the preparation of drugs to treat cerebral ischemia.

Further, the treatment is achieved by reducing the size of the cerebral infarction.

Further, the reduction of infarct size was achieved by down-regulating Glb1 expression to inhibit GM1 metabolism.

Further, the treatment is achieved by reducing cell damage and autophagy.

Further, the autophagy is the autophagy process of BV2 cells regulated by miR-6340/Glb1 through GM1 metabolism.

Further, the reduction of cell damage was achieved by down-regulating the expression of Glb1 to inhibit GM1 metabolism in microglia after OGD/R.

The second aspect of the invention provides a drug for treating cerebral ischemia, which contains miR-6340.

Glycosphingolipid biosynthesis-ganglioseries is a key metabolic pathway in stroke, and Glb1 is a key target in this metabolic pathway. This application locks the key target Glb1 for functional verification. The Glb1 gene encodes beta-galactosidase. Intracellular beta-galactosidase is located in organelles called lysosomes, which break down GM1 gangliosides and keratins sulfate.

In this application, by establishing a rat cerebral ischemia reperfusion model (MCAO/R) and an in vitro OGD/R model of microglia (BV2), microinjection of Glb1-siRNA (in vivo siRNA) in the right ventricle inhibited the target gene, and transfected BV2 cells with Glb1-siRNA in vitro. To study and analyze the molecular mechanism of whether Glb1 is involved in BV2 cell lesion during cerebral ischemia by regulating the synthesis of ganglioside. The miRNA of Glb1 protein coding gene (miR-6340) was predicted and verified, and the autophagy process of BV2 cells was studied through the regulation of mir-6340/Glb1 by GM1 metabolism.

This application shows the RNA expression levels of Glb1 gene in Human, pig and mouse brain regions in the HPA (Human Protein Atlas) database. The expression of target gene Glb1 in ischemic brain cells was determined by HE staining and immunofluorescence double staining of MCAO/R rat brain sections. After microinjection of GLB1-siRNA into the right ventricle of rats, RT-qPCR and ELISA experiments showed that down-regulating the expression of Glb1 could inhibit the metabolism of GM1 in MCAO/R rats and thus reduce the volume of cerebral infarction.

After establishing the OGD/R model of microglia in vitro, siRNA-Glb1 transfected microglia. RT-qPCR, WB, Hoechst staining, immunofluorescence assay and ELISA experiments showed that down-regulating the expression of Glb1 could inhibit the metabolism of GM1 in microglia after OGD/R and reduce cell damage.

This application further predicts the miRNA of the Glb1 protein-coding gene (miR-6340), which was confirmed by luciferase reporter gene assay. Molecular dynamics simulation showed that the Mir-6340-GLB1 ligand complex had high stability. RT-qPCR and WB experiments showed that mir-6340 negatively regulated the expression of Glb1 in microglia. ELISA showed that miR-6340/Glb1 was involved in the synthesis and metabolism of GM1 ganglioside in BV2 cells. mRNA levels of autophagy markers in BV2 cells were detected by RT-qPCR after Glb1 expression inhibition. It was further found by cell transmission electron microscopy that mir-6340/Glb1 regulated the autophagy process of BV2 cells through GM1 metabolism.

### BRIEF DESCRIPTION OF THE DRAWINGS

FIG. 1: Schematic diagram of brain stereotaxic experiment;

FIG. 2: mrna expression level of Glb1 gene in human brain region;

FIG. 3: mrna expression levels of Glb1 gene in two different mammalian brain regions, pig and mouse;

FIGS. 4A and 4B: Pathological detection of Glb1 protein in human tissues (FIG. 4A. Immunohistochemistry of Glb1 protein in human cerebral cortex. FIG. 4B. Double immunofluorescence staining of Glb1 protein and human glioma cells);

FIG. 5: histopathological changes of representative brain sections in rat MCAO/R model (100×);

FIGS. 6A to 6D: Immunofluorescence co-localization experiment after paraffin section;

FIG. 7: mRNA levels of Glb1 in rat brain tissue after detection of MCAO/R model by RT-qPCR (Note: n=6). ***P<0.001, ****P<0.0001);

FIG. 8: Representative MRI images of rat MCAO/R model after microinjection of Glb1-siRNA and NC-siRNA (Note: Compared with NC-siRNA group, **P<0.01, n=3, t test was used);

FIG. 9: The expression of GM1 in ischemic cerebral tissue of rats was detected by ELISA (note: * P<0.05, ***P<0.001, n=6).

FIGS. 10A and 10B: The expression of Glb1, p62, LC3II/I in ischemic cerebral tissue of rats was detected by WB test (Note: * P<0.05, ***P<0.001, n=3);

FIGS. 11A and 11B: BV2 cell viability after OGD/R model (Note: (FIG. 11A) Cell phenotype changes at different reoxygenation times after 3 h of hypoxia; (FIG. 11B) The effect of reoxygenation time on the survival rate of BV2 cells injured by hypoxia was determined by CCK-8 method, n=6);

FIG. 12: Iba1 expression was detected by immunofluorescence (Note: cells treated with OGD for 3 h and reoxygenated for 12 h).

FIG. 13: The effect of OGD/R on Glb1 expression in BV2 cells was detected by RT-qPCR (Note: OGD treated cells for 3 h and reoxygenated cells for 0 h, 6 h, 12 h and 24 h). n=3, **P<0.01, compared with normal group);

FIG. 14: The effect of OGD/R on Glb1 expression in BV2 cells detected by WB (Note: OGD treated cells for 3 h, and reoxygenated cells for 0 h, 6 h, 12 h and 24 h; n=4, **P<0.01, compared with normal group);

FIG. 15: Apoptosis was detected by Hoechst staining (Note: * P<0.05, **P<0.01, ***P<0.001, compared with the normal group);

FIG. 16: Detection of transfection efficiency of FAM-siRNA vector;

FIG. 17: The silencing efficiency of Glb1 was evaluated by RT-qPCR (Note: **P<0.01, ***P<0.001, compared with the normal group);

FIG. 18: OGD/R model was established after transfection, and Glb1 expression was detected by RT-qPCR (Note: Cells were treated with OGD for 3 h and reoxygenated for 12 h; **P<0.01, compared with normal group. ###P<0.001, compared with model group);

FIGS. 19A and 19B: mRNA levels of related enzymes in the synthesis and metabolism pathway of GM1 ganglioside detected by RT-qPCR (Note: (FIG. 19A) Schematic diagram of the synthesis and metabolism process of GM1; (FIG. 19B) mRNA levels of a series of enzymes involved in the GM1 synthesis and metabolism pathway; **P<0.01, ****P<0.0001, n=3);

FIGS. 20A to 20E: Molecular dynamics simulation experiment (Note: FIG. 20A. Free energy topography of Mir-6340-GLB1 complex-2D; FIG. 20B. Free energy topography of Mir-6340-GLB1 complex-3D; FIG. 20C. The number of intermolecular hydrogen bonds formed in the Mir-6340-GLB1 complex; FIG. 20D. RMSD map of mir-6340 and Glb1; FIG. 20E. RMSF map of Glb1 protein);

FIG. 21: Double luciferase reporting experiment (Note: * P<0.05);

FIG. 22: The effect of OGD/R on the expression of mir-6340 in BV2 cells was detected by RT-qPCR (Note: Cells were treated with OGD for 3 h and reoxygenated for 0 h, 6 h, 12 h and 24 h; n=6, *P<0.05, **P<0.01 compared with normal group);

FIG. 23: Effects of BV2 cells transfected with miRNA inhibitor and miRNA mimics on Glb1 expression detected by WB (Note: * P<0.05, ***P<0.001);

FIGS. 24A and 24B: Synthesis of GM1 ganglioside detected by Elisa (Note: FIG. 24A. Cell culture medium supernatant sample; FIG. 24B. Cell extract sample; * P<0.05, **P<0.01, ***P<0.001, ****P<0.0001);

FIG. 25: mRNA levels of autophagy markers were detected by RT-qPCR (Note: * P<0.05, **P<0.01, ***P<0.001, ****P<0.0001).

FIGS. 26A to 26E: Ultrastructure of BV2 cells (15000×, 1 μm) under transmission electron microscopy (Note: FIG. 26A: Control group; FIG. 26B: OGD/R group; FIG. 26C: OGD/R+mir-6340 inhibitor group; FIG. 26D: OGD/R+ siRNA-NC group; FIG. 26E: OGD/R+Glb1-siRNA).

### DETAILED DESCRIPTION OF THE EMBODIMENTS

The specific embodiments of the invention are described in detail below.

### 1. Experimental Materials and Methods

(1) Experimental animals: 30 SD male rats (SPF grade, weight 300±20 g) were purchased from Shanghai Slack Laboratory Animal Co., Ltd. The rats were raised in the Laboratory Animal Center of Zhejiang Chinese Medicine University with the experimental animal license number SYXK (Zhejiang) 2021-0012, with room temperature of 25±1° C., relative humidity of 50%-60%, light and dark cycle of 12 h/12 h, and free access to fresh water. All animals were conducted in strict accordance with the National Institutes of Health (NIH) Guidelines for the Care and Use of Laboratory Animals (Guidelines for the Care and Use of Laboratory Animals, 2011). The animal experiment program and guidelines were approved by the Animal Care and Use Committee of the Experimental Animal Research Center of Zhejiang Chinese Medical University.

Experimental group: \textcircled{1} Sham group (sham operation group), \textcircled{2} MCAO/R group (middle cerebral artery occlusion model group), \textcircled{3} MCAO/R+NC-siRNA group (MCAO/R+ right ventricular microinjection-no-loading control group), \textcircled{4} MCAO/R+Glb1-siRNA group (MCAO/R+right ventricular microinjection of Glb1-siRNA).
(2) Experimental Cells: The microglia cells used in the experiments of this application are derived from the 2nd generation BV2 cells (purchased from the Cell Bank of the Shanghai Institutes for Biological Sciences, Chinese Academy of Sciences).

Experimental Groups: \textcircled{1} Control Group (Normal Group), \textcircled{2} OGD/R Group (Oxygen-Glucose Deprivation/Reperfusion Model Group), \textcircled{3} OGD/R+inhibitor-NC Group (OGD/R+Inhibitor-Negative Control Group), \textcircled{4} OGD/R+miRNA inhibitor Group (OGD/R+miR-6340 inhibitor Group), \textcircled{5} OGD/R+NC-siRNA Group (OGD/R+Negative Control siRNA Group), \textcircled{6} OGD/R+Glb1-siRNA Group, \textcircled{7} OGD/R+miRNA inhibitor+Glb1-siRNA Group.

(3) Experimental Reagents

| Reagent Name | Brand |
|---|---|
| FastPure Cell/Tissue Total RNA Isolation Kit | Vazyme |
| 2X Universal SYBR Green Fast qPCR Mix | ABclonal |
| 5X All-In-One RT MasterMix | abm |
| Anti-Integrin beta 1 Antibody | abcam |
| GFAP Antibody | CST |
| NeuN Monoclonal Antibody | Proteintech |
| CD31/PECAM-1Antibody | SANTA CRUZ |
| Anti-Iba1 Antibody-Mouse IgG1 | abcam |
| Glb1Antibody | abcam |
| β-actin Antibody | CST |
| FITC-conjugated Goat Anti-Rabbit | ZSGB-BIO |
| IgG (H + L) | |
| Rhodamine-conjugated Goat | ZSGB-BIO |
| Anti-Mouse IgG (H + L) | |
| 0.25% Trypsin & 0.02% EDTA | Cienry |
| DMEM Glucose-Free Medium | BI |
| DMEM High-Glucose Medium | Gibco |
| CCK Cell Proliferation Assay Kit | ZOMANBIO |
| Total Protein Quantitation Assay Kit | Nanjing Jiancheng |
| (with Standards; BCA Method) | Bioengineering |
| | Institute |
| Serum-Free Cell Freezing Medium (CELLSAVING) | NCM Biotech |
| Primers | Sangon |
| (P1399) Poly-L-Lysine | Sigma |
| Cell Culture Slides - Diameter 15 mm | Biosharp |
| Fetal Bovine Serum | BI |
| Glutaraldehyde Fixative Solution | Yuanye |
| Anti-fluorescence quenching sealing solution | Beyotime |
| Glb1-siRNA | RiboBio |
| mir-inhibitor, mimics | RiboBio |
| MUT/WT Plasmid | Ruipute |
| Rat Anti-Ganglioside Antibody (GM1) | Camilo |
| ELISA Kit | |
| miRNA 1st Strand cDNA Synthesis Kit | Vazyme |
| miRNA Universal SYBR ® qPCR Master | Vazyme |
| Mouse Anti-Ganglioside Antibody (GM1) | RUIXIN TECH |
| ELISA Kit | |
| Dual Luciferase Reporter Gene Assay Kit | Yeasen |

(4) Experimental Equipment

| | |
|---|---|
| XP105 DeltaRange Analytical Balance | METTLER TOLEDO |
| CASCADA BIOMK2 Ultrapure Water System | PALL |
| Heraeus Fresco 21 Refrigerated | Thermo Fisher |
| Microcentrifuge | Scientific |
| NanoDrop One Microvolume UV-Vis | Thermo Fisher |
| Spectrophotometer | Scientific |
| Applied Biosystems QuantStudio 3 | Thermo Fisher |
| | Scientific |
| Kylin-Bell LX-600 Personal Microcentrifuge | Kylin-Bell |
| SCILOGEX D1008E Handheld Centrifuge | SCILOGEX |
| MICROM Cryostat Microtome | MICROM |
| OLYMPUS BX60 Fluorescence | OLYMPUS |
| Microscope Camera | |
| GNP-9080 Water-Jacketed Constant | Jinghong |
| Temperature Incubator | (Shanghai) |
| Fluorescence Inverted Microscope | Leica |
| Laminar Flow Cabinet or Biosafety Cabinet | Suzhou Purification |
| | Equipment |
| Vertical Plate Electrophoresis Cell | Bio-Rad |
| Electrophoresis Apparatus | Bio-Rad |
| Multimode Plate Reader | Thermo Fisher |
| | Scientific |

### 2. Experimental Method

### 2.1 Establishing the Rat MCAO/R Model

Select healthy adult Sprague-Dawley (SD) rats weighing approximately 250-300 g. Fix the rats in a supine position on the operating table and secure their limbs and head with tape. Use an electric razor to remove the hair on the midline of the rat's neck, from the lower jaw to the upper sternal notch. Disinfect the surgical area with iodine solution, lay sterile towels, and expose only the surgical field.

Neck incision and vessel exposure: Make a longitudinal incision of about 2-3 cm in the midline of the neck. Using ophthalmic scissors and forceps, carefully separate the subcutaneous tissue, bluntly dissect the muscle layer on both sides of the trachea, and expose the right common carotid artery (CCA), external carotid artery (ECA), and internal carotid artery (ICA). During the dissection, be cautious not to damage surrounding nerves, such as the vagus and sympathetic nerves, and minimize bleeding.

Artery ligation and preparation for filament insertion: First, ligate the distal end of the external carotid artery with silk suture, and leave a loose knot (slippery knot) at the proximal end. Then, temporarily clamp the common carotid artery and internal carotid artery with microvascular forceps to block blood flow. Make a small incision at the proximal end of the ligated external carotid artery, just large enough to insert the filament.

Filament insertion and positioning: Slowly insert the prepared filament through the incision in the external carotid artery, then gently pass the filament through the external carotid artery-common carotid artery bifurcation into the internal carotid artery. During insertion, maintain the correct direction of the filament and handle it gently. When the filament is inserted to a certain depth (generally about 18-20 mm, but the exact depth may vary slightly depending on the size of the rat and the vascular anatomy), you will feel slight resistance, indicating that the filament tip has usually reached the origin of the middle cerebral artery, blocking its blood flow and causing local cerebral ischemia. After that, tighten the loose knot on the external carotid artery to secure the filament.

Ischemic period: Maintain the filament insertion to keep the middle cerebral artery continuously ischemic. The ischemic time can be set according to the experimental purpose, generally 1-2 hours, to simulate different degrees of cerebral ischemic injury. During the ischemic period, closely monitor the rat's vital signs, such as respiration and heart rate, and maintain the rat's body temperature stable, which can be achieved using heating pads or similar devices to keep the rat's body temperature around 37° C.

Reperfusion period: After the predetermined ischemic time, carefully remove the filament, release the vascular clamps on the common carotid artery and internal carotid artery to restore blood flow, and begin the reperfusion phase. After reperfusion, also closely observe changes in the rat's neurological functions, such as limb activity and balance, as well as pathological and physiological changes in brain tissue.

2.2 H&E staining

### 2.2.1 Sample Pretreatment Steps

(a) Paraformaldehyde fixation of brain tissue: Five days after the MCAO/R model, anesthetize SD rats with Zoletil 50 (intramuscular injection, 40 mg/kg), expose the heart completely, insert the perfusion needle from the apex to the left ventricle, and quickly cut the left auricle to form a channel for the efflux of the perfusate. Rapidly push 100 mL of physiological saline through the syringe, followed by a slow and even push of 200 mL of 4% paraformaldehyde solution for fixation. After the perfusion is complete, quickly remove the intact brain and place it in 4% paraformaldehyde solution for fixation for 24 hours.
(b) Brain tissue fixation: Place the appropriately sized brain tissue in an embedding cassette and fix it in neutral buffered formalin (pH 7.4) for 2 hours; then place the fixed brain tissue in an automatic tissue processor for dehydration.
(c) Sample paraffin embedding: Place the transparent tissue blocks in melted paraffin, keep them in a paraffin melting box at 37° C. for 3 hours, and use 60-62° C. pathology sectioning paraffin for tissue embedding.
(d) Sample sectioning: Use a microtome to cut sections at a thickness of 4 μm, pick up the sections, and bake them in an oven at 60° C. for 2 hours.

### 2.2.2 HE Staining Procedure

Deparaffinize the prepared sections in xylene for 10 minutes, twice, followed by gradient ethanol solutions of 100%, 95%, 80%, and 70% to remove paraffin, until the surface is free of water. After rehydration, place the sections in hematoxylin staining solution for 10 minutes, and rinse with distilled water for 2 minutes. Dehydrate the processed sections in 95% ethanol for 3 minutes, twice, and then stain with 0.5% alcoholic eosin solution for 2 minutes. Dehydrate the sections with 80% ethanol and 95% ethanol, each for 3 minutes, twice, followed by 100% ethanol for 3 minutes, twice, and clear in xylene for 5 minutes, twice. Mount and observe the sections under a light microscope for pathological characteristics of brain tissue, and capture images.

### 2.3 Immunofluorescence Double Staining Experiment

### 2.3.1 Sample Pretreatment Steps: Same as Steps 2.1

### 2.3.2 Immunofluorescence Detection Procedure

(a) Sample deparaffinization and rehydration: The pre-treated samples are deparaffinized in xylene for 10 minutes, twice, and then subjected to a gradient ethanol series of 100%, 95%, 80%, and 70% for deparaffinization, followed by rehydration with distilled water.
(b) Antigen retrieval: Place the sections in a retrieval solution of 0.1 mol/L citrate buffer at pH=6, and heat in a microwave at medium power for 2 minutes followed by a pause for 1 minute, then heat at medium-low power for another 2 minutes. After stopping the heating, let the sections stand for 15 minutes, and then wash with PBS solution for 5 minutes, three times in total. After that, place the sections in a 3% H₂O₂ solution for 10 minutes, and wash again with PBS solution for 5 minutes, three times in total.
(c) Primary antibody incubation: Use an immunohistochemistry pen to circle the sections (the pen should not touch water). Apply appropriately diluted primary antibodies from two different species sources to the sections, with the addition of 0.3% Triton X-100, and incubate in a humid chamber at 4° C. overnight.
(d) Fluorescent secondary antibody incubation: Wash with PBS solution for 5 minutes, three times in total. Apply fluorescent secondary antibodies from two different species sources to the washed sections, and incubate at 37° C. for 60 minutes. Wash with PBS solution for 5 minutes, three times in total.
(e) DAPI staining: Mount the sections with an anti-fade reagent containing DAPI.
(f) Microscopic observation: Observe the number of positive cells in brain tissue sections under the microscope after the samples have been processed.
   2.4 Intracerebroventricular Injection of Glb1-siRNA in Rats (In Vivo siRNA)

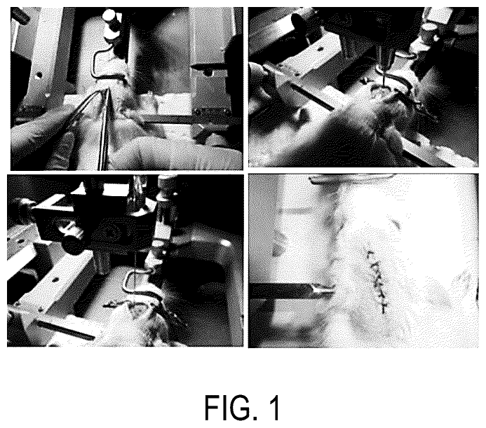

Rats are fasted for 12 hours before surgery and anesthetized with zoletil 50 (intramuscular injection, 40 mg/kg). Hair on the head is removed using an electric shaver. An incision is made along the midline of the rat's head, and the fascia is dissected until the intersection of the coronal suture and sagittal suture (the anterior fontanelle) is fully exposed. According to the rat brain stereotactic atlas, locate the right lateral ventricle of the rat, which is 1.5 mm lateral to the anterior fontanelle, 1.1 mm in the direction of the posterior fontanelle, and 4.5 mm deep (coordinates are ±1.5, −1.1, −4.5), and mark the injection point with a pencil. The rats are randomly divided into the NC-siRNA group and the Glb1-siRNA group, with the NC-siRNA group serving as a negative control. The injection is performed slowly at a rate of 2 μL/min, with a total injection volume of 10 μL, containing 10 nmol. The needle is left in place for 10 minutes after the injection. After the surgery, the scalp is disinfected and sutured. Both groups of rats are prepared with the MCAO/R model using the thread method, with all other operations being consistent. FIG. 1 shows a simplified flowchart of the brain stereotactic experiment.

### 2.5 Magnetic Resonance Imaging (MRI) for Detecting Cerebral Infarct Volume in Rats

On the fifth day after model establishment, rats from each group were anesthetized with zoletil 50. The rats were placed prone on the scanning bed with their heads fixed. Magnetic Resonance Imaging (MRI) measurements were conducted using a 7 Tesla 20 cm bore superconducting magnet (Magnex Scientific Inc.) interfaced with a Bruker Biospin MRI console. A 3-plane scout imaging sequence was used to adjust the position of the animal's head, with a repetition time TR of 3500 ms, echo time TE of 102 ms, and a scanning thickness of 2 mm, covering the entire brain of the rat. The inherent atomic nuclei within the animal's body, under the influence of an external magnetic field, produce resonance, absorb energy, and emit MR signals. These signals are collected and used as the source for imaging, processed by a computer to form MR images of the animal's body, and to obtain T2-weighted images (T2WI) of the cerebral infarction site. Image J image analysis software was used to measure the volume of cerebral infarction in rats.

### 2.6 Reverse Transcription Quantitative Polymerase Chain Reaction (RT-qPCR) Experiment

According to the instructions of the FastPure® Cell/Tissue Total RNA Isolation Kit V2, brain tissues from each group of rats were ground and lysed thoroughly on ice, followed by multiple washes to remove impurities, and total RNA was extracted from the samples. Using the 5×All-In-One RT MasterMix kit, a 20 μL reverse transcription reaction system was used to transcribe total RNA into cDNA, with the reaction program: 25° C. for 10 minutes; 42° C. for 15 minutes; 85° C. for 5 minutes.

The Applied Biosystems QuantStudio 3 software system was used, with primer sequences as shown in Table 1. A 20 μL amplification system was employed: 10 μL Universal SYBR Green Fast qPCR Mix, 0.4 μL forward primer (10 μM), 0.4 μL reverse primer (10 μM), 2 μL cDNA, and 7.2 μL ddH2O for real-time PCR analysis. The PCR thermal cycling steps were as follows: initial denaturation at 95° C. for 3 minutes; cycling reaction at 95° C. for 5 seconds and 60° C. for 30 seconds for 40 cycles; melting curve analysis at 95° C. for 15 seconds; 60° C. for 1 minute; 95° C. for 15 seconds. The PCR products were analyzed using the melting curve to confirm the correctness of the amplification. Based on the obtained CT values, with GAPDH as the reference gene and the sham surgery group as the control group, the relative expression levels of 10 each gene's mRNA were calculated using the formula:

2^{-ΔΔCT}=2^{\hat{ }[(Experimental Group Target Gene CT-Experimental Group Reference Gene CT)-(Control Group Target Gene CT-Control Group Reference Gene CT)}] Calculation formula:

TABLE 1
Primer Sequence Table

| Gene | | |
|---|---|---|
| | Rat Primer Sequences | |
| Glb1 | F: AAGTTCAAGACG | R: CCAGTTGGTGAGGA |
| | GTGACGGAAGC | CAGTGAAGTTG |
| | (SEQ ID NO: 1) | (SEQ ID NO: 2) |
| Gapdh | F: GTCCATGCCATC | R: CGCCTGCTTCACCA |
| | ACTGCCACTC | CCTTCTTG |
| | (SEQ ID NO: 3) | (SEQ ID NO: 4) |
| | Mouse Primer Sequences | |
| Glb1 | F: ATGAAGATGGCT | R: CAGGAAGCGTAG |
| | GGGCTGAATGC | GTAGTCGTAATCG |
| | (SEQ ID NO: 5) | (SEQ ID NO: 6) |
| Gapdh | F: TCACCATCTTCC | R: TGAGCCCTTCCA |
| | AGGAGCGAGAC | CAATGCCAAAG |
| | (SEQ ID NO: 7) | (SEQ ID NO: 8) |
| ATG7 | F: CCCAGAAGAAGTT | R: GCAGACCAGCAG |
| | GAACGAGTACCG | AGTCACCATTG |
| | (SEQ ID NO: 9) | (SEQ ID NO: 10) |
| LC3A | F: GAGCGAGTTGGT | R: CATAGATGTCAG |
| | CAAGATCATCCG | CGATGGGTGTGG |
| | (SEQ ID NO: 11) | (SEQ ID NO: 12) |
| LC3B | F: CAAGCCTTCTTC | R: CCATTGCTGTCC |
| | CTCCTGGTGAATG | CGAATGTCTCC |
| | (SEQ ID NO: 13) | (SEQ ID NO: 14) |
| Bcn1 | F: CGCAAGGTGGTG | R: ACTCACTATACT |
| | GCAGAGAAC | CCCGCTGGTACTG |
| | (SEQ ID NO: 15) | (SEQ ID NO: 16) |
| LAMP1 | F: GGTTTGGGTCTG | R: AGGTAGGCAATG |
| | TGGAAGAGTGTG | AGGACGATGAGG |
| | (SEQ ID NO: 17) | (SEQ ID NO: 18) |
| LAMP2 | F: CCAACTCCAACT | R: GGCACCTTCTCCT |
| | CCAACTCCAACC | CAGTGATGTTC |
| | (SEQ ID NO: 19) | (SEQ ID NO: 20) |
| GM1 | F: TCCTCGGCCTCT | R: CTTACACCACGG |
| | GATTCCTCTTG | CACGAACTCTC |
| | (SEQ ID NO: 21) | (SEQ ID NO: 22) |
| GM2 | F: GCACAGGCAACT | R: CCCTTGAGAGAG |
| | ACCGCATCC | GCAGCAATCTTG |
| | (SEQ ID NO: 23) | (SEQ ID NO: 24) |
| Hexa | F: GTCACTCACATC | R: AATTCTGCCAGC |
| | TACACAGCACAGG | ACACGGATACC |
| | (SEQ ID NO: 25) | (SEQ ID NO: 26) |
| GALC | F: CTTCAGGACATG | R: ATAGGATCGTGC |
| | CGGACGTTACAG | CGTTCAACATACC |
| | (SEQ ID NO: 27) | (SEQ ID NO: 28) |
| GM3 | F: GAGGACAGGTAC | R: AAGTCATGTTCG |
| | AGCATCAACTTGG | GGCAGCATATCC |
| | (SEQ ID NO: 29) | (SEQ ID NO: 30) |
| NEU1 | F: CGCAGCTATGAC | R: AGGGTGGGCTGG |
| | GCCTGTGAC | ATTGGAGAAG |
| | (SEQ ID NO: 31) | (SEQ ID NO: 32) |
| GBA | F: AAGGGGTCGCTC | R: GCTGTAGGTTCA |
| | AAGGGTCAG | TTCTCCGCTGTC |
| | (SEQ ID NO: 33) | (SEQ ID NO: 34) |

### 2.7 Western Blot Experiment (WB)

(1) Gel preparation: Based on the molecular weight of the target protein, sequentially prepare a 12.6% separating gel and a 5% stacking gel.
(2) SDS-PAGE electrophoresis: Prepare the electrophoresis buffer, with a sample load of 10-20 μg, and add an equal volume of quantified protein sample to each well. Cover the electrophoresis tank, connect to the power supply, and run the electrophoresis at 80 V for 30 minutes, followed by 120 V for 80 minutes. Electrophoresis buffer: Weigh 1.0 g SDS, 14.40 g Glycine, and 3.00 g Tris-base powder, add ddH2O and stir to fully dissolve, and finally make up to 1 L.
(3) Transfer: Prepare the transfer buffer, activate the PVDF membrane with methanol, and transfer the proteins onto the PVDF membrane. Place the black side (cathode) at the bottom, followed by a sponge, filter paper, gel, PVDF membrane, then another layer of filter paper, sponge, and the white side (anode). Electrophoresis conditions: 0.3 A for 2 hours. Place the electrophoresis tank in a container with ice for electrophoresis. 10×Transfer Buffer stock solution: Weigh 144.13 g Glycine and 30.28 g Tris-base powder, add ddH2O and stir to fully dissolve, and finally make up to 1 L; 1×Transfer Buffer: Take 50 mL of 10×Transfer Buffer stock solution, add 100 mL methanol and 350 mL ddH2O, mix well before use.
(4) Blocking: Prepare 5% skim milk with 1×TBST and block the PVDF membrane on a shaker for 1 hour. 1×TBST solution: Weigh 8.70 g NaCl, add 250 mL 2 M Tris-HCl and 1 mL Tween 20, add ddH2O and stir to mix well, and finally make up to 1 L.
(5) Primary antibody incubation: Dilute the primary antibody (1:1000) according to the antibody manual with antibody dilution buffer, and incubate the PVDF membrane with the primary antibody overnight at 4° C.
(6) Secondary antibody incubation: Quickly wash the PVDF membrane twice with 1×TBST and then wash it again three times. Each wash lasts for 10 minutes. Dilute the HRP-conjugated secondary antibody (1:5000) with 1×TBST solution and incubate at room temperature for 1 hour.
(7) Development: Wash the PVDF membrane three times with 1×TBST solution, each wash lasting for 10 minutes. Gently add the development mixture to the PVDF membrane, incubate at room temperature for 1 minute, then transfer the PVDF membrane to the development instrument, and subsequently perform quantitative analysis with ImageJ software.

### 2.8 Enzyme-Linked Immunosorbent Assay (ELISA) for Detecting the Expression of Gangliosides

(1) Preparation of standard samples: Allow the freeze-dried rat GM1 standard to stand for 5 minutes and then centrifuge slowly. Afterward, add 1.0 mL of standard dilution solution and let it dissolve completely, then mix gently (concentration is 20 ng/ml), and label the tube as \textcircled{1}. Take 7 centrifuge tubes and label them as \textcircled{2}, \textcircled{3}, \textcircled{4}, \textcircled{5}, \textcircled{6}, \textcircled{7}, \textcircled{8}. Add 300 μL of standard dilution solution to each tube. Transfer 300 μL from tube \textcircled{1} to tube \textcircled{2} and mix well, then transfer 300 μL from tube \textcircled{2} to tube \textcircled{3}, and continue this process until tube \textcircled{7}. Tube \textcircled{8} is the standard dilution solution and serves as the blank group. The final concentrations in each tube are 20, 10, 5, 2.5, 1.25, 0.625, 0.312 ng/mL, respectively, to be used as the standard curve.
(2) Sample detection: Add 100 μL of each group's sample or different concentrations of rat GM1 standard (0 ng/mL well with standard dilution solution) to the microplate; incubate at 37° C. for 90 minutes.
(3) Wash the plate with wash solution twice. Add 100 μL of rat GM1 antigen working solution to each well (prepare the rat GM1 antigen working solution 30 minutes in advance). Incubate at 37° C. for 60 minutes.
(4) Wash the plate three times. Add 100 μL of enzyme conjugate working solution to each well except for the blank well, which should be prepared in the dark 30 minutes in advance. Seal the reaction wells with adhesive tape and incubate in the dark at 37° C. for 30 minutes.
(5) Wash the plate with wash solution five times. Add 100 μL of TMB chromogenic working solution to each well (including the blank well), and incubate in the dark at 37° C. Terminate the reaction when the high concentration of the standard curve shows a deep color with a clear color gradient.
(6) Add 100 μL of stop solution to each well, including the blank well, mix well, and measure immediately with a microplate reader set to OD value 450 nm.

### 2.9 Cell Culture

### 2.9.1 Cell Thawing

(1) Prepare a 15 mL sterile centrifuge tube and add 8 mL of culture medium.
(2) Remove the cryovial from the liquid nitrogen container and immerse it in 37° C. warm water, occasionally shaking to ensure rapid thawing.
(3) Wipe the biosafety cabinet with a cotton ball soaked in 75% alcohol, remove the cryovial from the 37° C. water bath, and transfer the cells from the cryovial to the prepared centrifuge tube containing 8 mL of culture medium. Gently pipette to evenly disperse the cells.
(4) Centrifuge at 1,000 rpm for 5 minutes. Discard the supernatant, add 1 mL of fresh culture medium, and gently pipette to create a cell suspension.
(5) Count the cells, adjust the cell density, and seed into culture flasks. Transfer the cell suspension to a T25 culture flask, add culture medium up to 5 mL, and gently rock the flask to ensure even cell distribution. Place the flask in a 37° C. incubator for static culture.
(6) Observe the cell attachment and growth, and replace the culture medium once the next day.

### 2.9.2 Cell Passage

(1) Passage the cells when they reach 80% to 90% confluence. Aspirate the old culture medium from the flask and wash the cells with PBS solution 2 to 3 times.
(2) Add an appropriate amount of trypsin digestion solution to the flask and gently shake the culture flask to ensure the digestion solution covers all cell surfaces.
(3) Incubate in the incubator for 50 seconds. When you observe cell body retraction and an increase in the gaps between cells, immediately terminate the digestion by adding an equal volume of complete culture medium to stop the digestion.
(4) Aspirate the culture medium from the flask, repeatedly pipette the cell wall to detach the cells from the flask, forming a cell suspension. Transfer the cells to a 15 mL centrifuge tube and centrifuge at 1,000 rpm for 5 minutes.
(5) Discard the supernatant, resuspend the cells in pre-warmed complete culture medium, count the cells, dilute the cell suspension to the appropriate seeding density, transfer to new cell culture flasks, and place them in the cell culture incubator for further culture.

### 2.9.3 Cell Cryopreservation

(1) When the cell confluence reaches 80% to 90%, proceed with cell cryopreservation. Following the same trypsinization steps as mentioned earlier for passaging, digest the monolayer-grown cells with trypsin, centrifuge to obtain a cell pellet.
(2) Add an appropriate amount of cryopreservation medium and gently pipette to ensure even cell distribution. After adjusting the cell concentration, transfer 1 mL of cell suspension into a cryovial. Label the cryovial with the cell line name, date of cryopreservation, and the operator's name.
(3) Place the cryovials in a −80° C. freezer overnight, and finally transfer them to liquid nitrogen for long-term storage.

### 2.10 Glb1-siRNA Transfection

(1) Seed BV2 cells at a density of 30×10\hat{ }4 cells per well into a 6-well plate containing an appropriate amount of complete culture medium (v1).
(2) Dilute siRNA: Dilute 5 μL of 20 μM siRNA stock solution (v3) with 120 μL of 1×riboFECT™ CP Buffer (v2), and gently mix.
(3) Preparation of the complex: Add 12 μL of riboFECT™ CP Reagent (v4), gently pipette to mix, and incubate at room temperature for 15 minutes to prepare the transfection complex.
(4) Add the riboFECT™ CP transfection complex to an appropriate amount of serum-free complete culture medium (v1), and gently mix.
(5) Place the culture plate in a 37° C. CO₂ incubator and culture for 24 hours.

### 2.11 Establishing an Oxygen-Glucose Deprivation/Reperfusion (OGD/R) Cell Model

Cells are cultured in DMEM medium containing 10% fetal bovine serum and 1% penicillin-streptomycin solution, and incubated in a 37° C. incubator with 5% CO₂ to establish the OGD/R model with hypoxia for 3 hours. When the cells grow to cover 80% of the area of the culture dish, remove the culture medium and wash the cells with PBS. Then, add serum-free and glucose-free DMEM to the cells and place them in a homemade hypoxia box, seal it, and culture for 3 hours to induce hypoxia in the cells. Control group BV2 cells are cultured in a normal incubator. BV2 cells are treated with reperfusion using normal culture medium with 10% serum concentration, and reoxygenation times are 0 h, 6 h, 12 h, and 24 h.

### 2.12 Cell Viability Experiment

Cells are seeded into a 96-well plate, and after OGD/R treatment, cell viability is assessed using the Cell Counting Kit-8 (CCK-8) according to the manufacturer's instructions. Add 10 μL of CCK-8 solution to each well and incubate at 37° C. for 1 hour. The optical density (OD) values are measured at a wavelength of 450 nm. The cell survival rate (%)=(OD value of OGD/R−OD value of blank well)/(OD value of normal control group-OD value of blank well)×100%.

### 2.13 Hoechst Staining for Detecting Apoptosis

Hoechst 33342, a DNA-binding dye, is used to detect apoptosis in BV2 cells. After treatment under OGD/R conditions, cells are fixed in 4% paraformaldehyde at room temperature for 10 minutes and then incubated with 10 μg/mL Hoechst 33342 in the dark at 37° C. for 25 minutes. The cells are then washed three times with PBS and observed immediately under a fluorescence microscope. Cells exhibiting strong blue fluorescence and nuclear condensation are scored as apoptotic. The percentage of apoptosis is calculated by the ratio of apoptotic cells to the total number of counted cells.

### 2.14 Dual Luciferase Reporter Experiment

(1) BV2 cells are routinely cultured under conditions of 37° C. and 5% CO₂.
(2) BV2 cells in the logarithmic growth phase are seeded into a 96-well plate at a density of 1.0×10\hat{ }4 cells per well with a total volume of 100 μL per well, and cultured in a 37° C. incubator for 24 hours.
(3) Take 5 μL of serum-free medium to dilute miRNA mimics or Non-target Control, and the target gene 3′UTR dual-reporter gene vector or mutant vector, and 5 μL of medium to dilute 0.25 μL Lipo6000™ transfection reagent. Let each stand for 5 minutes, then mix gently and let stand for another 5 minutes. The final concentration of mimics is 50 nM, and the amount of plasmid used is 100 ng. Set up three replicate wells for each group, and replace the culture medium 6 hours after transfection.
(4) After 48 hours of transfection, aspirate the culture medium and add 300 μL of lysis buffer. Incubate on ice for 5 minutes to lyse the cells.
(5) Take 20 μL of the lysed sample and add it to the culture plate. Set up three replicate wells as needed for the experiment.
(6) Dilute the firefly luciferase substrate (50×) and the renilla luciferase substrate (50×) to 1× working solution with the corresponding buffer. Prepare in advance, protect from light, and incubate at room temperature.
(7) Add 100 μL of firefly luciferase reaction solution, mix by shaking the plate, and measure the activity of firefly luciferase, which should be completed within 30 minutes.
(8) Add 100 μL of renilla luciferase reaction solution, mix by shaking the plate, and measure the activity of renilla luciferase, which should be completed within 30 minutes. The ratio of firefly fluorescence signal values to renilla fluorescence signal values is used as the final value for each group.

### 2.15 Transmission Electron Microscopy (TEM) Observation of Autophagy in Cells

(1) After digesting BV2 cells, neutralize them with culture medium or serum and transfer them into a 1.5 mL centrifuge tube, centrifuge for 3 minutes at 1000 rpm. Gently aspirate the supernatant along the wall of the tube, add 500-1000 μL of 5% serum, disperse the cells by pipetting, and then centrifuge.
(2) Gently aspirate the supernatant, keeping the sample in a clumped state, add 1 mL of PBS and wash once.
(3) Remove the supernatant, then add 1 mL of 2.5% glutaraldehyde solution (the volume must be more than 20 times the volume of the cell clump; if it appears turbid, it should not be used). You can use a toothpick to scrape around the wall of the tube to separate the cells from the wall (about the size of a grain of millet), ensuring that each cell clump is in full contact with the fixative, and fix at 4° C. for more than 4 hours.
(4) After fixation with 2.5% glutaraldehyde, rinse with PBS, postfix with 1% osmium tetroxide, dehydrate gradually with acetone, soak in Epon-812 embedding agent, and then embed and cure. Ultrathin sections (70 nm) are double-stained with uranyl acetate and lead citrate. Observe the changes in autophagic structures in cells under a transmission electron microscope.

### 2.16 Statistical Analysis of Data

Experimental data are expressed as the mean±standard deviation (SD) and statistically analyzed using GraphPad Prism 8.0.1 software. Comparisons between two groups are performed using a t-test for significance analysis. Comparisons among multiple groups are analyzed using one-way analysis of variance (ANOVA) or non-parametric tests. A P-value less than 0.05 indicates that the data is statistically significant.

### 3. Experimental Results

### 3.1 the Impact of Glb1, a Common Target of the Three Categories of Formulas, on Microglia in the Brain

### 3.1.1 HPA Database

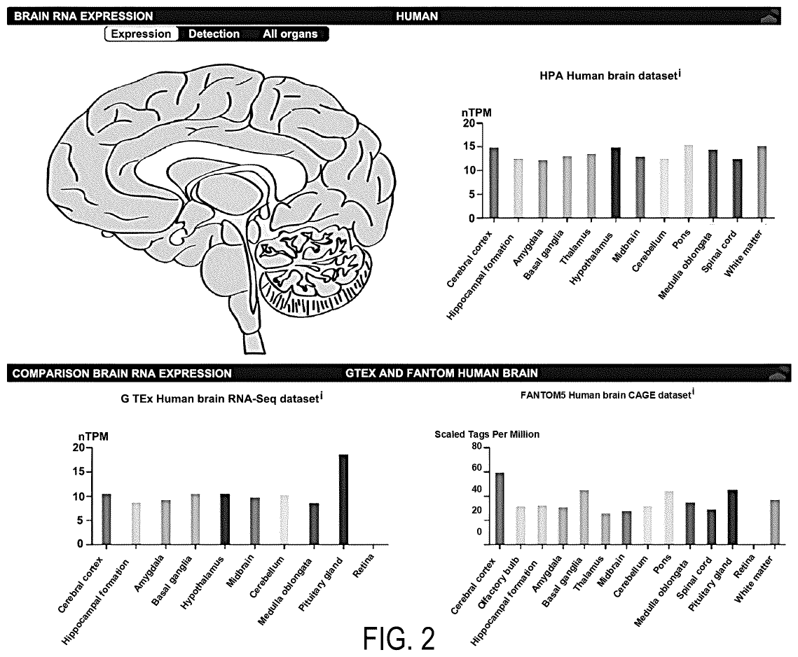

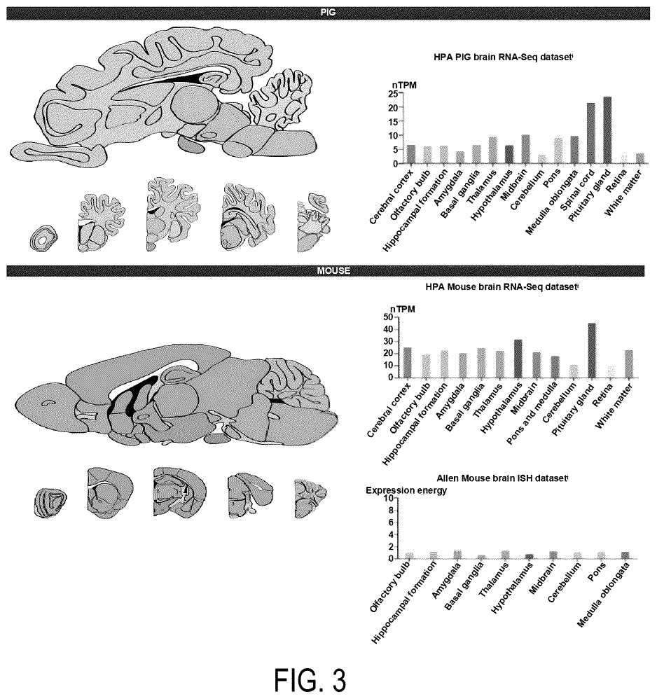

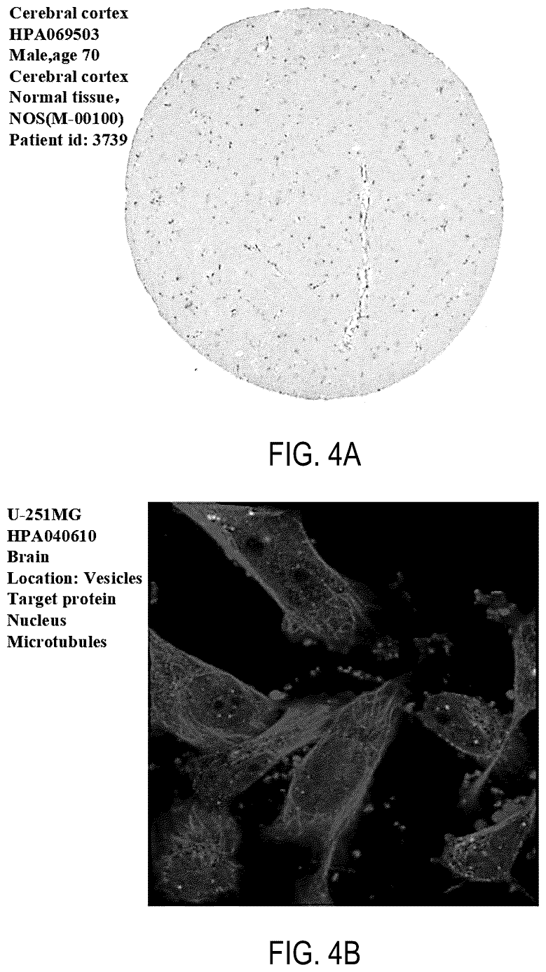

To investigate the expression levels of the Glb1 protein in human tissues and cells, this part of the study utilized data from the Human Protein Atlas (HPA) database, specifically the Brain Atlas and pathological section data. The HPA database is a freely accessible public database that contains over 26,000 antibodies targeting more than 17,000 human genes. The Brain Atlas, which integrates data from humans, pigs, and mice, demonstrates the RNA expression of different proteins within the brains of these mammalian species. RNA-seq data generated by the Genotype-Tissue Expression (GTEx) project show that Glb1 is primarily expressed in the pituitary, retina, and cerebral cortex in the human brain; in the pituitary, hypothalamus, and cerebral cortex in the mouse brain; and in the spinal cord, pituitary, and midbrain in the pig brain (FIGS. 2-3, note: images sourced from the HPA database at https://www.proteinatlas.org/ENSG00000170266-GLB1). Pathological section results from human brain tissues indicate that Glb1 has high expression in the cerebral cortex, and immunofluorescence results from human glioma cells (U-251 MG) also show expression of Glb1 (FIGS. 4A and 4B, image sourced from the HPA database at https://www.proteinatlas.org/ENSG00000170266-GLB1).

### 3.1.2 Histopathological Changes in Brain Sections of the Rat MCAO/R Model

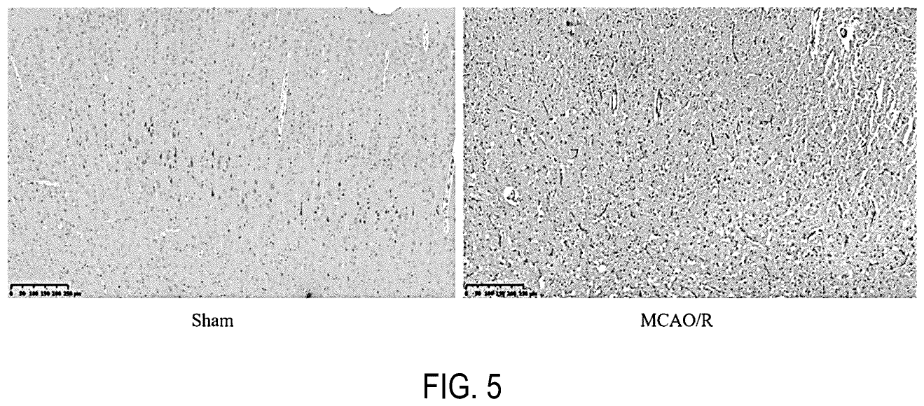

After the MCAO/R model, blood was taken from the abdominal aorta, and the brain was perfused with 4% paraformaldehyde. The abdominal aorta was clamped, and a perfusion needle was inserted through the left ventricle and fixed. The right auricle was cut open, and first, 100 mL of physiological saline was rapidly perfused, followed by a slow and even perfusion of 200 mL of 4% paraformaldehyde. After taking the brain, it was placed in 4% paraformaldehyde for later use. The fixed brain slices, after going through the steps of dehydration and embedding, were prepared into paraffin continuous sections, and 4 μm thickness sections were taken for HE staining. HE staining showed that in the normal group, cells were intact and arranged neatly. In the MCAO/R rats, the brain tissue around the cortex of the top leaf of the brain was loosely edematous, nuclei were vacuolated, and there were demyelination changes around (FIG. 5).

### 3.1.3 Expression of Common Targets in Brain Cells of the Ischemic Hemisphere in Rats

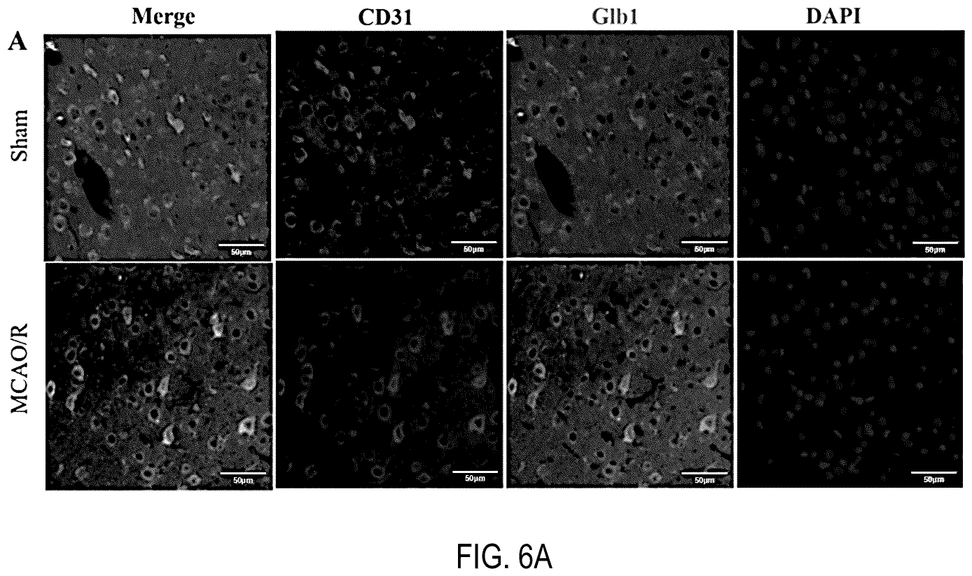

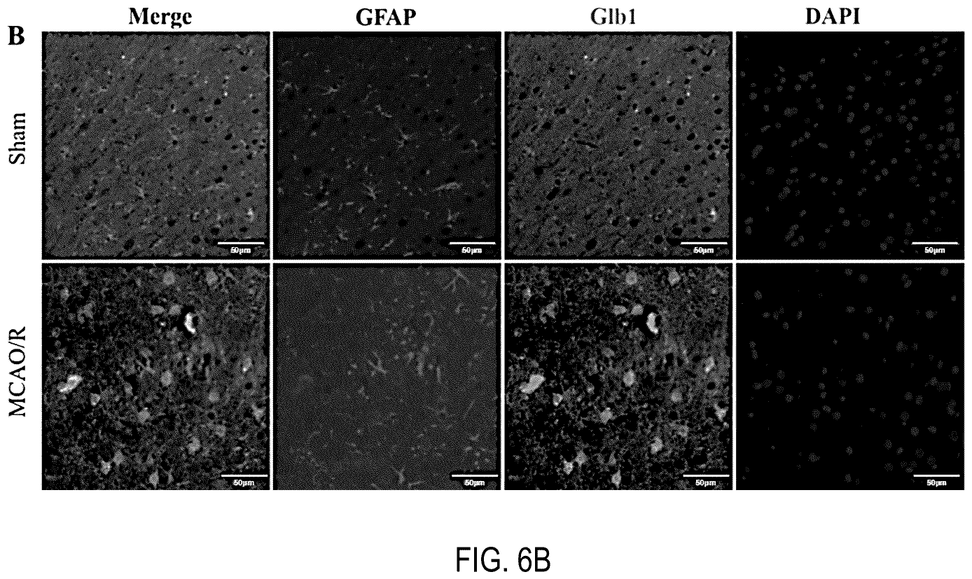

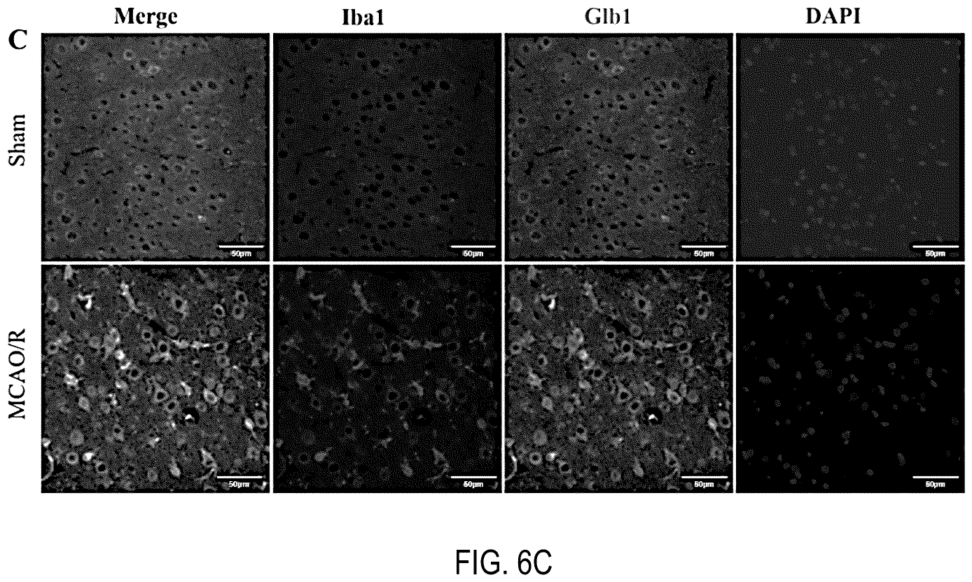

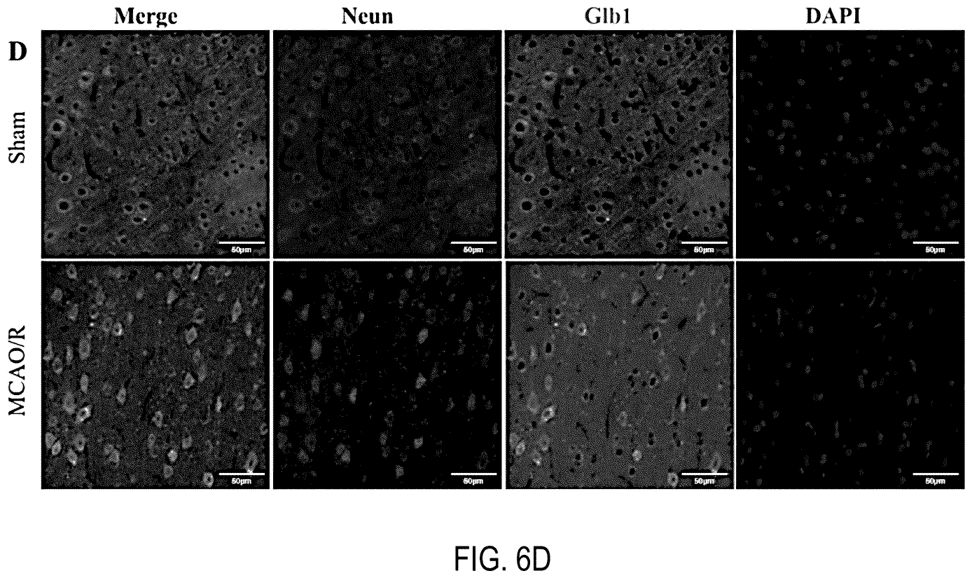

Immunofluorescent double staining of rat MCAO/R model brain paraffin sections was performed using endothelial cell, astrocyte, microglia, and neuronal cell markers along with Glb1. The number and phenotype of marked cells in the MCAO/R model group were compared. The results shown in FIGS. 6A to 6D indicate that the co-localization of Iba1 with Glb1 is significantly higher than that of other markers, suggesting that Glb1 is highly expressed in microglia after modeling. Therefore, microglia were chosen for subsequent experiments.

### 3.2 Downregulating the Expression of Glb1 can Inhibit the Metabolism of GM1 in MCAO/R Rats

3.2.1 Expression of Glb1 after Intracerebroventricular Injection of Glb1-siRNA

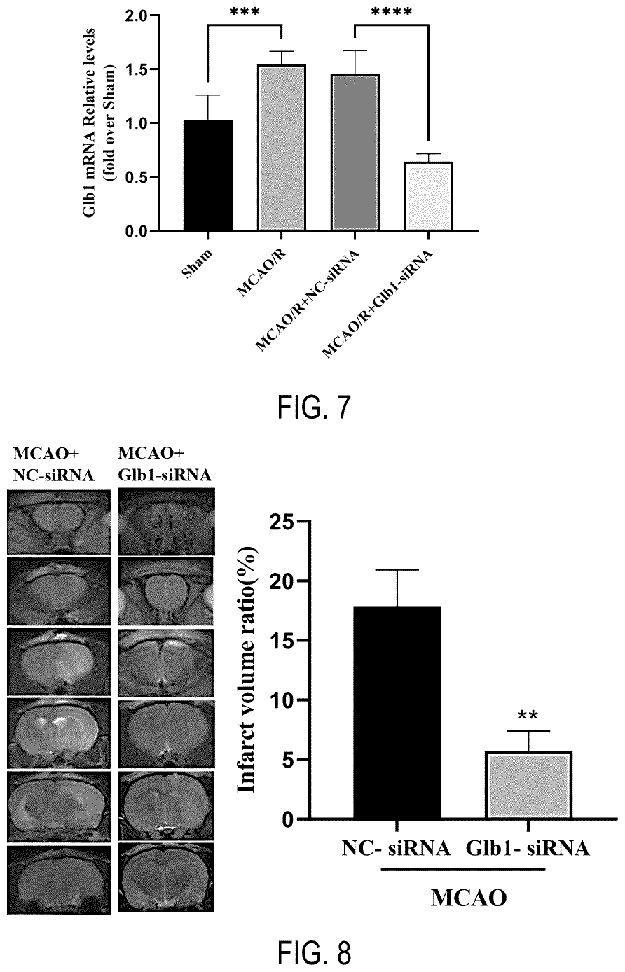

After intracerebroventricular injection of Glb1-siRNA (in vivo siRNA) and NC-siRNA in rats, to detect the effectiveness of siRNA transduction, the present application detected the expression of Glb1 in the right lateral ventricle of the rat brain through RT-qPCR experiments. Compared with the Sham group, the mRNA level of Glb1 in the MCAO/R group was significantly increased (P<0.001); compared with the MCAO/R group, there was no significant change in the mRNA level of Glb1 in the MCAO/R+NC-siRNA group, indicating that the NC-siRNA negative control had no effect on the expression of Glb1 in rats; compared with the MCAO/R+NC-siRNA group, the mRNA level of Glb1 in the MCAO/R+Glb1-siRNA group was significantly decreased (P<0.0001), indicating that the inhibitory efficiency of in vivo injection of Glb1-siRNA is high, and subsequent experiments can be conducted (FIG. 7).

### 3.2.2 Inhibition of Glb1 Expression can Reduce the Infarct Area in MCAO/R Rats

After intracerebroventricular injection of Glb1-siRNA and NC-siRNA in rats, both groups were subjected to MCAO/R modeling. Five days later, magnetic resonance imaging (MRI) was used to assess the infarct area and detect the extent of brain injury. As shown in FIG. 8, the infarct volume in rats microinjected with Glb1-siRNA was significantly reduced (P<0.01).

### 3.2.3 Inhibition of Glb1 Expression can Suppress GM1 Metabolism in MCAO/R Rats

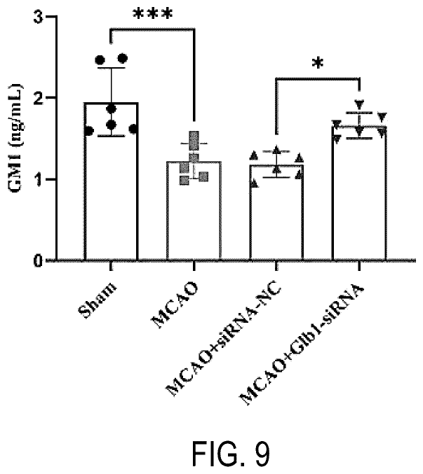

Weigh out 0.05 g of tissue and add 500 μL of PBS at pH 7.2. Homogenize the tissue thoroughly with a homogenizer, centrifuge, and collect the supernatant. The expression of GM1 in each group was detected by Elisa experiment, and the results are shown in FIG. 9. Compared with the Sham group, the expression of GM1 in the MCAO/R group was significantly decreased (P<0.001); compared with the MCAO/R group, there was no significant change in GM1 levels in the MCAO/R+NC-siRNA group, indicating that the NC-siRNA negative control had no effect on the expression of GM1 in rats; compared with the MCAO/R+NC-siRNA group, the expression level of GM1 in the MCAO/R+Glb1-siRNA group was significantly increased (P<0.05), suggesting that inhibiting the expression of Glb1 can suppress the metabolism of GM1 in MCAO/R rats.

### 3.2.4 Inhibition of Glb1 Expression can Reduce the Level of Autophagy in MCAO/R Rats

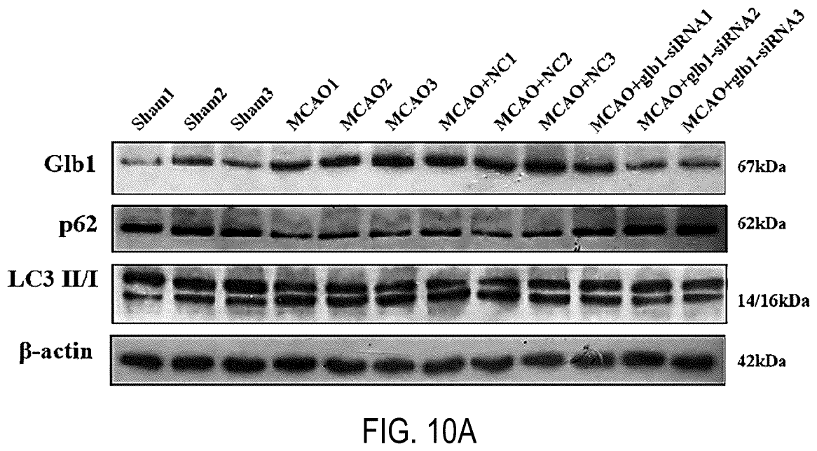

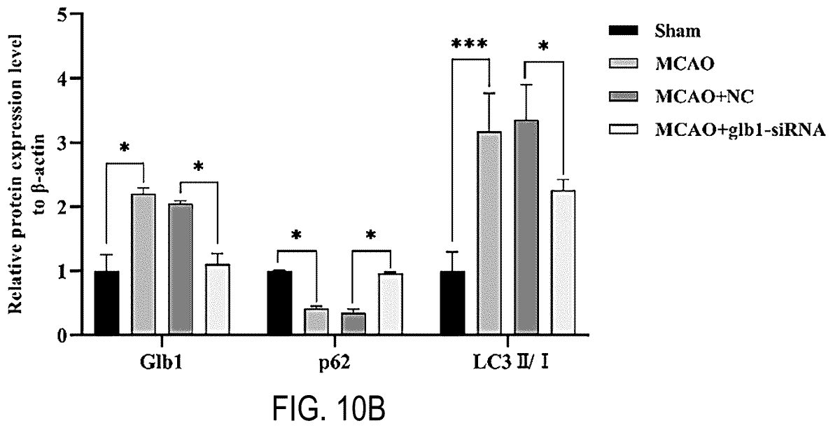

Through literature review, it has been found that oxidative stress culture shows an increase in aging-related β-galactosidase (Glb1/SAβ-gal), leading to significant changes in the lysosomal system, including increased lysosomal mass and the content of autophagic vacuoles, and the accumulation of oxidized materials and damaged mitochondria within lysosomes. Therefore, this application used WB experiments to detect the expression of autophagy markers p62 and LC3II/I in the brain tissue of the ischemic side of rats. As shown in FIGS. 10A and 10B, compared with the Sham group, the expression level of Glb1 in the MCAO group was significantly increased (P<0.05), the expression level of p62 was significantly decreased (P<0.05), and the expression level of LC3II/I was significantly increased (P<0.001). Compared with the MCAO group, there were no significant changes in the expression levels of Glb1, p62, and LC3II/I in the MCAO+NC-siRNA group; compared with the MCAO+NC-siRNA group, the expression level of Glb1 was significantly decreased (P<0.05), the expression level of p62 was significantly increased (P<0.05), and the expression level of LC3II/I was significantly decreased (P<0.05) in the MCAO+Glb1-siRNA group.

### 3.3 Downregulating the Expression of Glb1 can Inhibit GM1 Metabolism in Microglia After OGD/R

3.3.1 the Trend of BV2 Cell Phenotype Changes Over Time with Reperfusion after OGD/R Model

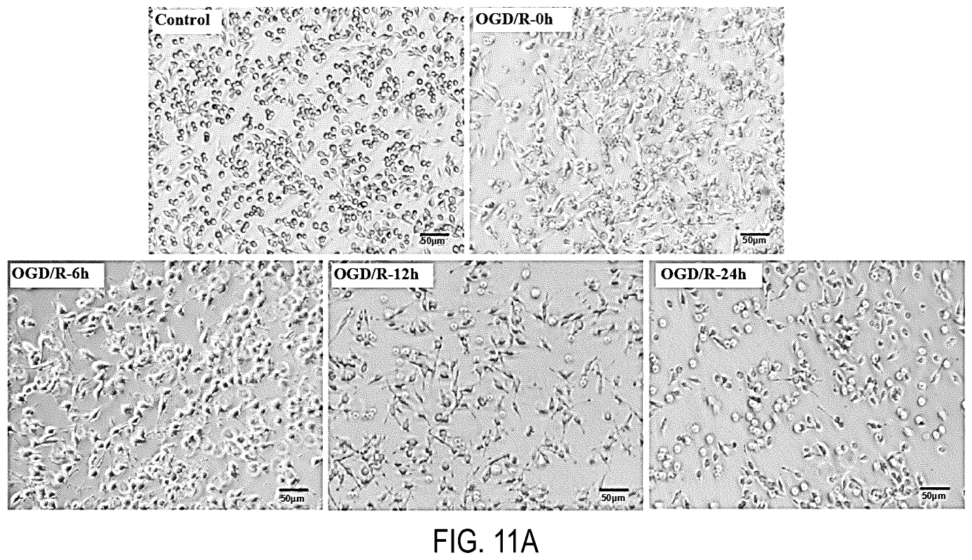

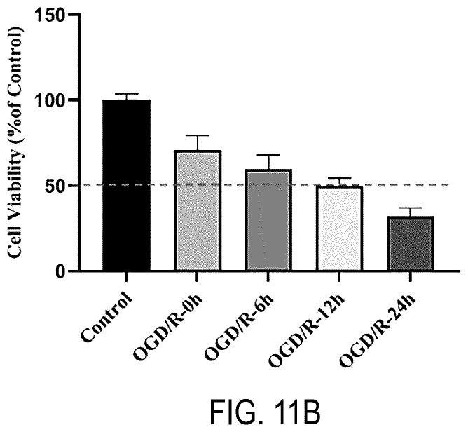

Control group BV2 cells are cultured in a normal incubator. BV2 cells are treated with reperfusion using normal culture medium with a 10% serum concentration, with reoxygenation times of 0 h, 6 h, 12 h, and 24 h. The optimal reoxygenation time is selected through CCK8 assay. With 3 hours of hypoxia, the optimal reoxygenation time is 12 h, which is used for subsequent mechanism studies (FIGS. 11A and 11B).

3.3.2 BV2 Cells are Activated after OGD/R

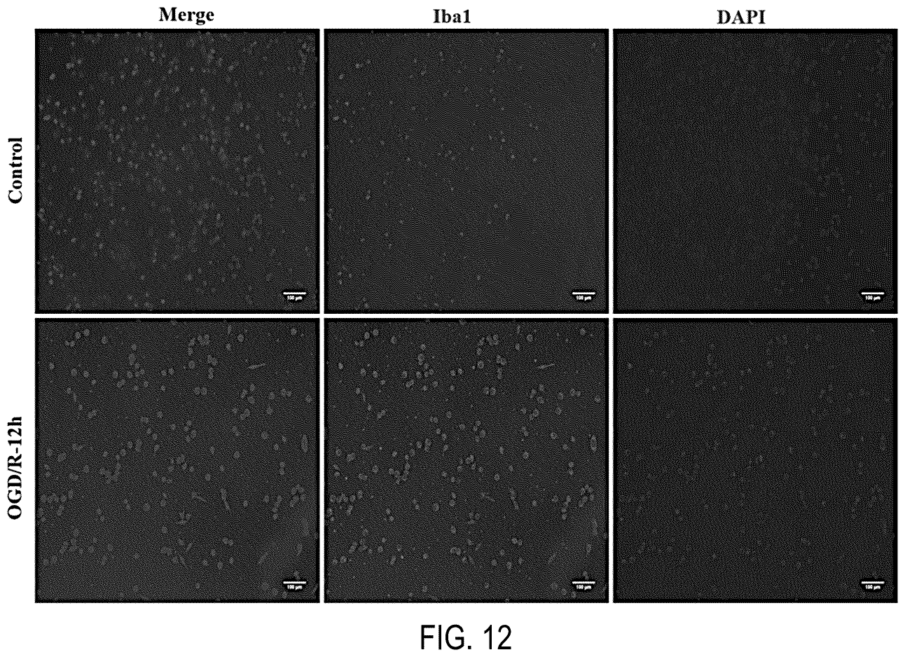

Microglia were subjected to OGD/R and treated with reperfusion at 0 h, 6 h, 12 h, and 24 h. Fixed with 4% paraformaldehyde at room temperature for 30 minutes; blocked with 1% BSA for 1 hour, then incubated with the primary antibody (Iba1 1:200) overnight at 4° C.; the next day, after washing with PBS, the fluorescent secondary antibody (1:1000) was labeled for 1 hour, and DAPI was used to stain the nuclei for 5 minutes; the slides were mounted and scanned using a fluorescence microscope. The immunofluorescence results further confirmed that after 12 hours of reoxygenation following OGD/R, the expression of the microglia activation marker Iba1 significantly increased, indicating that the microglia were activated (FIG. 12).

3.3.3 Expression Levels of Glb1 in BV2 Cells after the OGD/R Model

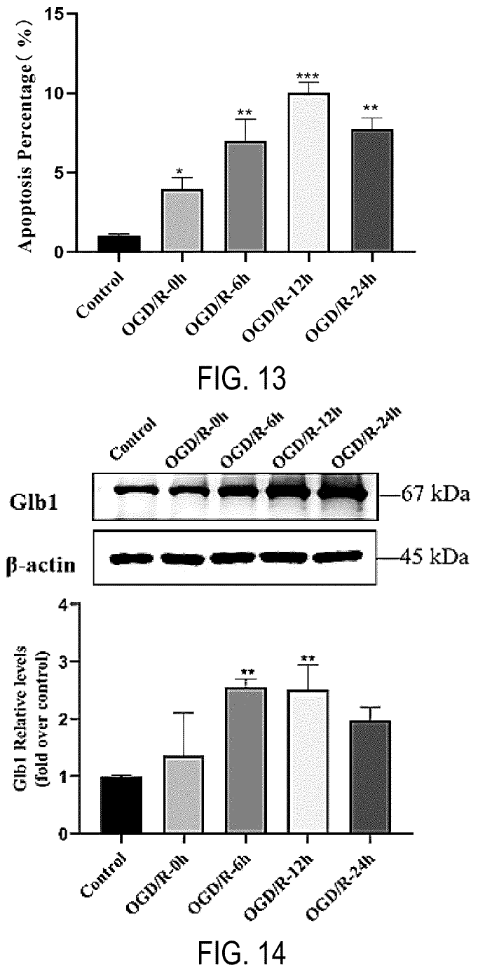

Total RNA from BV2 cells was extracted using the FastPure Cell/Tissue Total RNA Isolation Kit V2 kit. RNA was reverse transcribed into cDNA using a reverse transcription kit. Subsequently, real-time fluorescence quantitative PCR detection was performed using 2× Universal SYBR Green Fast qPCR Mix. The experiment was repeated three times. Glyceraldehyde-3-phosphate dehydrogenase (Gapdh) was used as a reference gene for the control experiment, and the relative expression levels were analyzed using the 2−ΔΔCt method. The real-time fluorescence quantitative PCR results (FIG. 13) indicated that, compared with the control group, the mRNA expression levels of Glb1 were upregulated in microglia after OGD/R at reoxygenation times of 0 h, 6 h, and 12 h (P<0.01). Western-Blot results are shown in FIG. 14, and compared with the normal group, the protein expression levels of Glb1 were significantly increased after OGD/R-6 h and OGD/R-12 h (P<0.01).

3.3.4 Hoechst Staining for Detecting Cell Apoptosis after the OGD/R Model

The OGD/R model was established, and Hoechst staining was used to detect cell apoptosis. As shown in FIG. 15, the normal group cells have a uniform distribution of genomic DNA, and after Hoechst staining, they appear as a uniform blue, with the cell nuclei being circular. Apoptotic cells, due to nuclear condensation and fragmentation, have nuclei that appear crescent-shaped or fragmented into several pieces under Hoechst staining, presenting a bright white-blue color. Compared with the normal group, OGD/R-12 h showed a decrease in cell proliferation and a significant increase in apoptotic cells (P<0.001).

### 3.3.5 Glb1-siRNA Transfection of Microglia

### 3.3.5.1 Transfection Efficiency

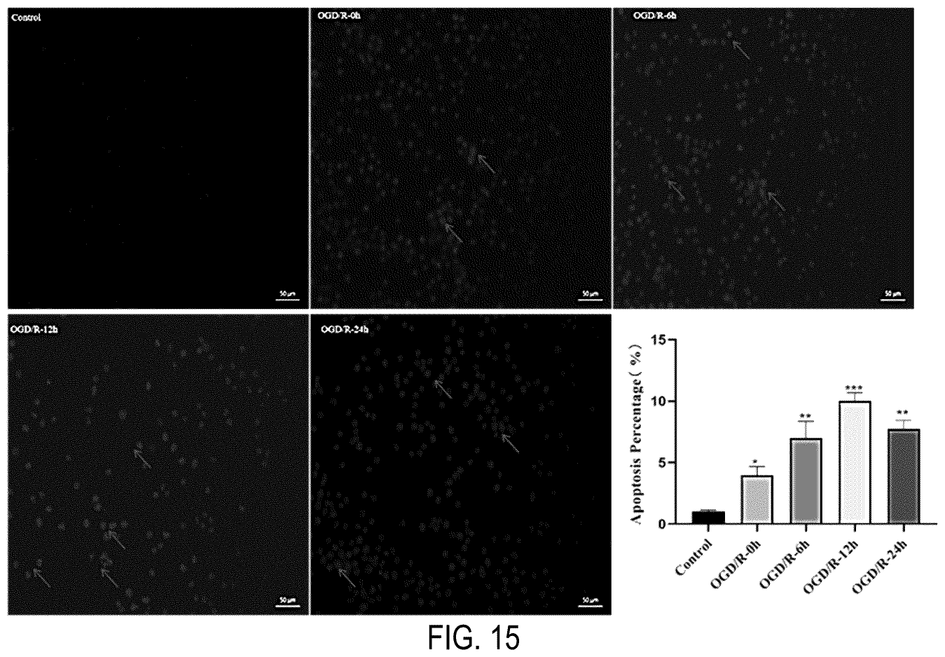

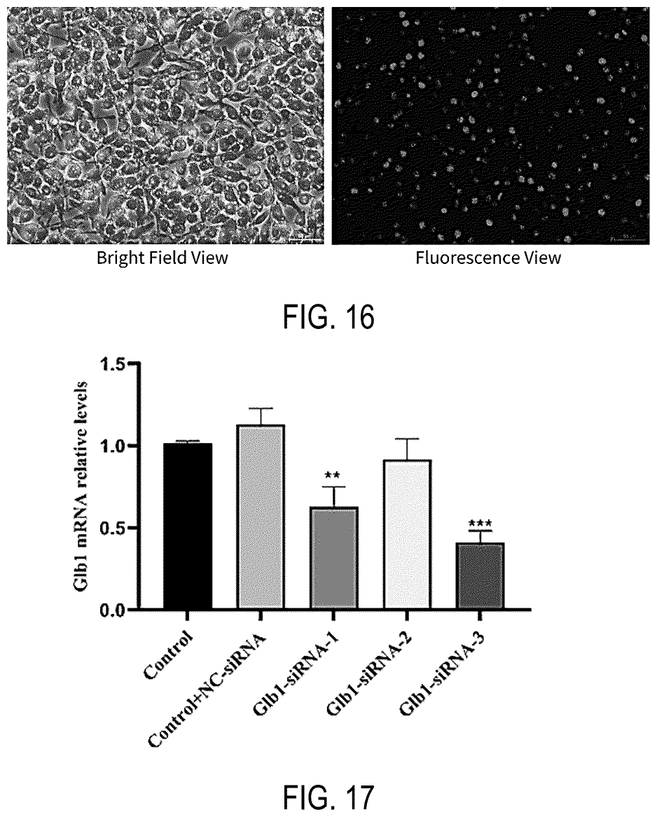

To detect the transfection efficiency of siRNA, the green fluorescence of FAM-siRNA under a fluorescence microscope was compared with the field of view under a brightfield microscope (counting the number of cells under both white light and fluorescence). As shown in FIG. 16, the time point with the highest siRNA transfection efficiency was 24 hours post-transfection, with a siRNA concentration of 100 nM, and the transfection efficiency of the cells had reached over 80%, making it suitable for proceeding with subsequent experiments.

3.3.5.2 Expression of Glb1 in BV2 Cells after Transfection with Glb1-siRNA

In this experiment, three siRNAs targeting Glb1 and a negative control NC-siRNA were synthesized, with a final transfection concentration of 100 nM siRNA. The efficiency of Glb1 silencing was determined by RT-qPCR to select the most efficient Glb1-siRNA for subsequent mechanism studies. BV2 cells were divided into five groups: (1) Normal group, (2) Normal group+NC-siRNA, (3) Glb1-siRNA-1, (4) Glb1-siRNA-2, (5) Glb1-siRNA-3. The expression of Glb1 after transfection was detected by RT-qPCR. As shown in FIG. 17, we observed that Glb1-siRNA-3 exhibited the best interference efficiency (P<0.001), and the treated cells were labeled as the Glb1-siRNA group.

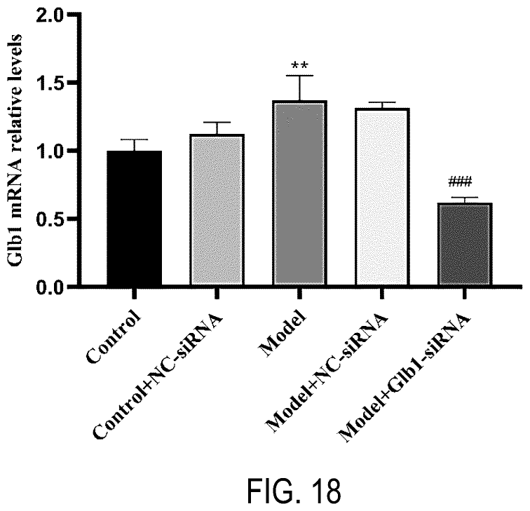

After transfection, the OGD/R model was established at 24 hours, and the expression of Glb1 was detected by RT-qPCR. BV2 cells were divided into five groups: (1) Control, (2) Control+NC-siRNA, (3) Model group, (4) Model+NC-siRNA group, (5) Model+Glb1-siRNA group. As shown in FIG. 18, compared with the normal group, the mRNA level of Glb1 in the model group was significantly increased (P<0.01); compared with the model group, there was no significant change in the mRNA level of Glb1 in the Model+NC-siRNA group, indicating that the NC-siRNA negative control had no effect on the expression of Glb1 in BV2 cells; compared with the Model+NC-siRNA group, the mRNA level of Glb1 in the Model+Glb1-siRNA group was significantly decreased (P<0.001), indicating that the inhibitory efficiency of in vivo injection of Glb1-siRNA was high, and subsequent experiments could be conducted.

### 3.3.6 Downregulating the Expression of Glb1 can Inhibit GM1 Metabolism in BV2 Cells

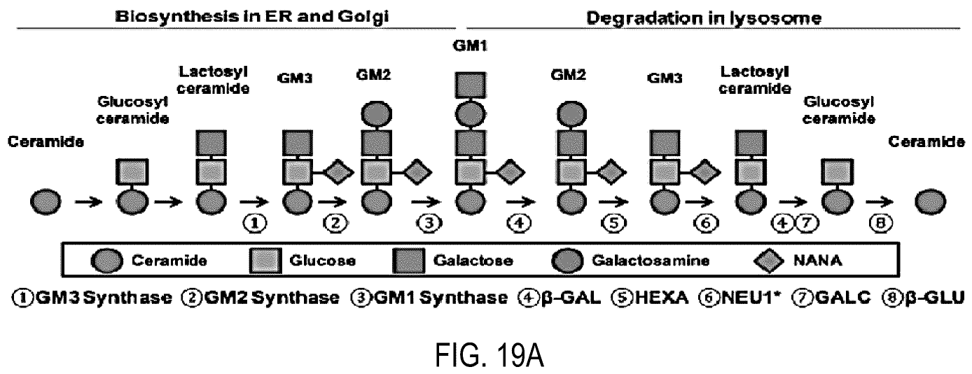

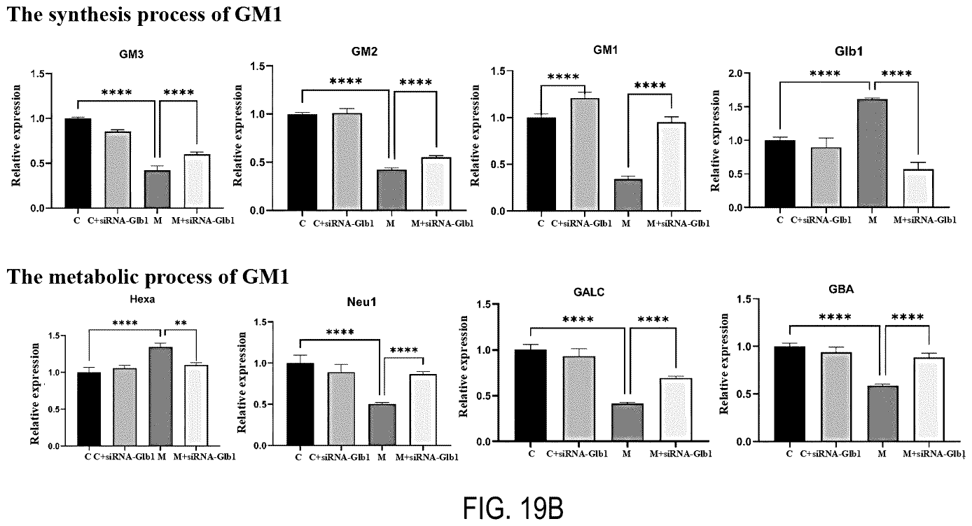

After transfection with Glb1-siRNA for 24 hours, the OGD/R model was established, and the mRNA levels of a series of enzymes involved in the synthesis and metabolism of GM1 ganglioside were measured by RT-qPCR, including the biosynthetic enzymes GM3, GM2, and GM1, as well as the mRNA expression levels of Glb1, Hexa, Neu1, GALC, and GBA during GM1 metabolism. The results shown in FIGS. 19A and 19B indicate that, compared with the normal group, the expression levels of GM3, GM2, and GM1, which are involved in GM1 biosynthesis, were significantly reduced in the OGD/R group, suggesting a weakened GM1 synthesis pathway; at the same time, after transfection with Glb1-siRNA, the expression levels of GM3, GM2, and GM1 all showed an upward trend. Compared with the normal group, the expression of Glb1 and Hexa in the GM1 metabolism pathway was increased, while the mRNA expression levels of Neu1, GALC, and GBA were decreased in the OGD/R group, indicating an initial enhancement followed by a weakening of the GM1 metabolism pathway; after transfection with Glb1-siRNA, the expression levels of Glb1 and Hexa were weakened, and the mRNA expression levels of Neu1, GALC, and GBA all showed a downward trend, indicating a weakened GM1 metabolism process. 3.4 miR-6340/Glb1 Involves in the Metabolism of GM1 Ganglioside in BV2 Cells

3.4.1 Prediction of Target Gene Glb1's miRNA and Molecular Dynamics Simulation of miR-6340-Glb1

To elucidate the molecular mechanisms by which target genes regulate the activation of BV2 cells in ischemic stroke, miRNAs were screened through three databases: TargetScan, MiRDB, and miRWalk. The software analysis results indicated that there is a high possibility of binding between the 3′ untranslated region (3′UTR) of Glb1 mRNA and mmu-miR-6340, with a binding site present.

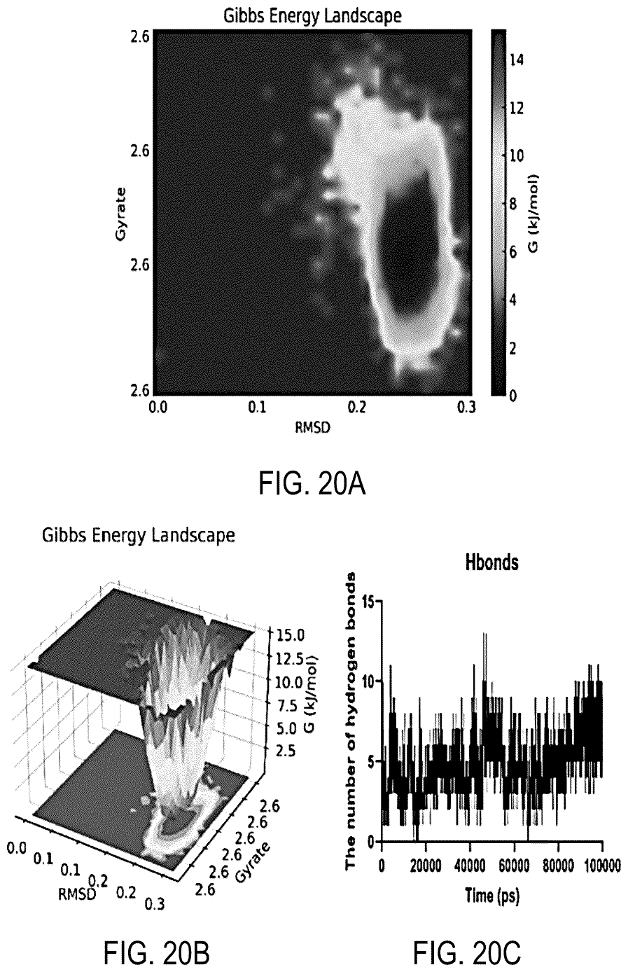

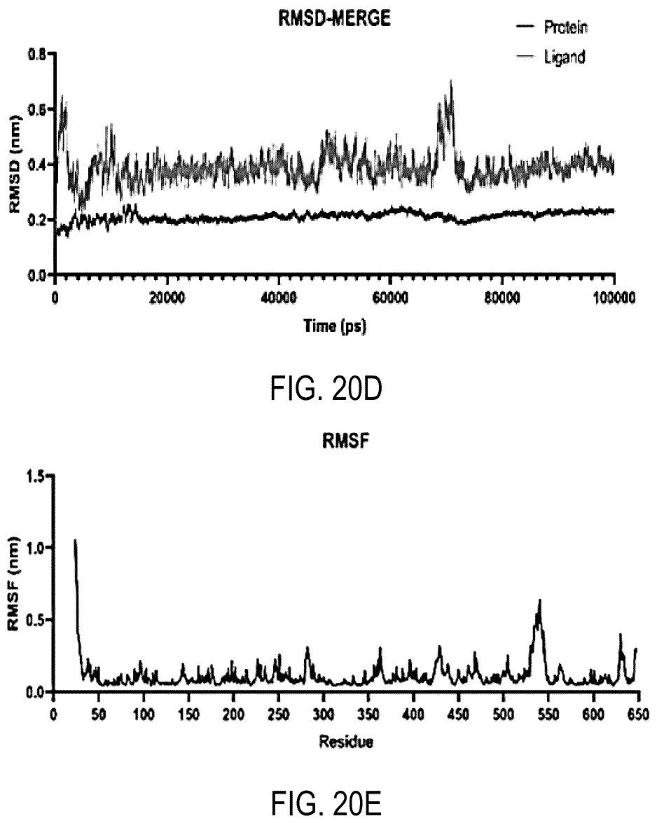

To further verify the interaction between Glb1 and miR-6340, this application conducts molecular dynamics simulation experiments, which are based on the fundamental principles of Newtonian mechanics and utilize computer software to simulate the interactions and motion changes of macromolecules. Trajectory sampling starting from different polypeptide conformations yields a free energy landscape (Gibbs Energy Landscape) (FIGS. 20A and 20B). It displays the valleys and hills of free energy that the polypeptide experiences throughout the simulation process, with blue indicating lower energy and red indicating higher energy. RMSD (Root Mean Square Deviation) measures the average distance between atoms of two different conformational proteins, and the trend of RMSD for proteins and ligands is also an important characteristic for determining whether the simulation has reached stability. RMSF (Root Mean Square Fluctuation) measures the deviation of atomic positions over time relative to a reference position, characterizing the flexibility and intensity of motion of protein amino acids throughout the simulation process. Hbonds is a hydrogen bond analysis tool. FIG. 20C calculates the number of hydrogen bonds in the ligand-protein complex, and hydrogen bonds between amino acids can alter the internal geometry of proteins. FIG. 20D shows that the RMSD value of the Glb1 protein is low, indicating high stability of the protein over the simulation time. Meanwhile, the small molecule miR-6340 ligand remains relatively flat after an initial fluctuation. Amino acids close to the active site usually exhibit lower RMSF values, and the results in FIG. 20E show that the RMSF values of the Glb1 protein are low, indicating a higher ligand-protein interaction over the simulation time. In summary, the molecular dynamics simulation experiment further validates the stable and reliable binding between miR-6340 and Glb1.

### 3.4.2 Dual Luciferase Reporter Gene

The Dual Luciferase Reporter Gene Assay confirms the interaction between miRNA and target genes by downregulating the relative fluorescence values of the reporter genes. The pmirGLO dual luciferase reporter vector uses the sea pansy luciferase gene (hRluc) as the reporter fluorescence and the firefly luciferase gene (hluc) for normalization. The 3′UTR region of the gene is cloned downstream of the hRluc gene; miRNA acts on target genes through the 3′UTR region, so miRNA is co-transfected with the constructed reporter gene vector. The ratio of firefly fluorescence signal values to sea pansy fluorescence signal values is used as the final value for each group.

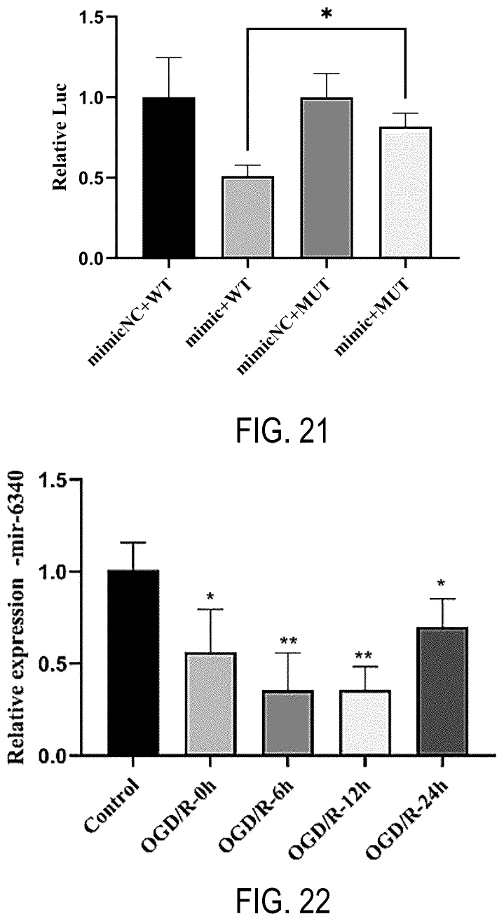

As shown in FIG. 21, the results of the luciferase reporter gene assay indicate that, after normalization with the NC group, co-transfection with miR-6340 mimic led to an increase and recovery in the reported fluorescence of Glb1-MUT pmirGLO compared to Glb1-WT pmirGLO (Note: * P<0.05). This result suggests that miR-6340 interacts with the 3′UTR site of Glb1.

3.4.3 OGD/R Treatment Leads to Downregulation of miR-6340 Expression in BV2 Cells

Reverse transcription was performed using the stem-loop method for miRNA cDNA synthesis and to detect the level of miR-6340. Table 2 lists the primer sequences and stem-loop sequences. The RT-qPCR results showed that compared with the control group, the expression level of miR-6340 in BV2 cells treated with OGD/R for 0 h, 6 h, 12 h, and 24 h was significantly downregulated (P<0.05), with the lowest levels reached at 6 h and 12 h after reperfusion (FIG. 22).

TABLE 2
miR-6340 Primer sequence

| Gene | Primer sequence |
|---|---|
| miR-6340 | F: 5′-ACAACAACGTCAGCAGCAGCT-3′ |
| | (SEQ ID NO: 35) |
| | R: 5′-CAGTGCGTGTCGTGGAGT-3′ |
| | (SEQ ID NO: 36) |
| Stem-Loop | 5′-GTCGTATCCAGTGCAGGGTCCGAGGTA |
| Sequence | TTCGCACTGGATACGACGCCAAA-3′ |
| | (SEQ ID NO: 37) |
| Internal | F: 5′-ATCCAGTGCAGGGTCCGAGG-3′ |
| ControlU6 | (SEQ ID NO: 38) |
| | R: 5′-AACGCTTCACGAA TTTGCGT-3′ |
| | (SEQ ID NO: 39) |

3.4.4 miR-6340 Regulates Glb1 Expression

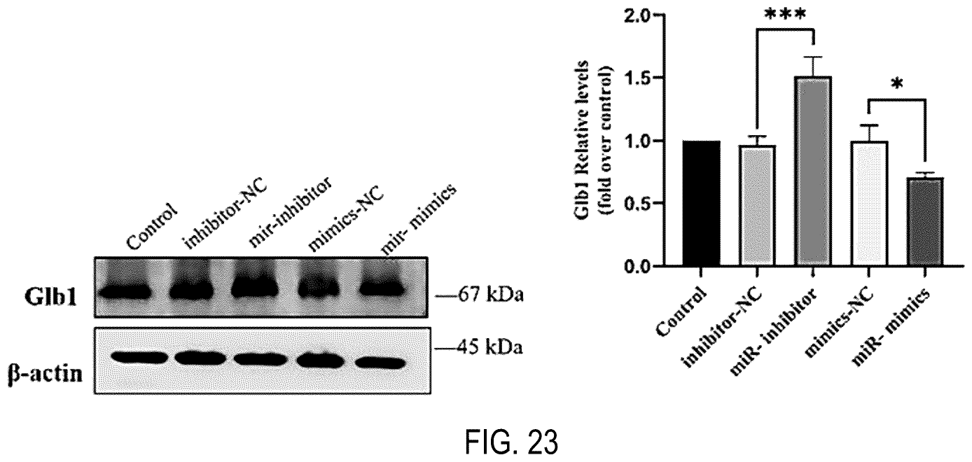

miRNA inhibitors and mimics were synthesized using chemical methods to suppress and enhance the regulatory effects of miR-6340, respectively. Cell suspensions containing miR-6340 inhibitor/inhibitor-NC and miR-6340 mimics/mimics-NC were transferred to a 6-well cell culture plate at a transfection concentration of 100 nM. After 48 hours of culture, cells were collected for subsequent Western blotting experiments to detect the expression of the target gene Glb1. The Western blotting experiments showed (FIG. 23), compared with the inhibitor-NC group, the miR-6340 inhibitor group upregulated the expression of Glb1 in microglia (P<0.001); compared with the mimics-NC group, the overexpression of miR-6340 mimics downregulated the expression of Glb1 in microglia (P<0.05). The results indicated that miR-6340 negatively regulates the expression of Glb1 in BV2 cells.

3.4.5 miR-6340/Glb1 Participates in the Synthesis and Metabolism of GM1 Ganglioside in BV2 Cells

Glb1-siRNA and miR-6340 inhibitor were co-transfected into microglia. Microglia were plated and cultured for 24 hours before the medium was changed and the cells were transfected with miR-6340 inhibitor and Glb1-siRNA. After 24 hours of culture, the cells were subjected to OGD/R treatment. The culture medium from each group was collected, and the supernatant was carefully collected after centrifugation for 20 minutes. Following the protocol outlined in the kit's instructions, the relative expression of GM1 ganglioside in the cell culture supernatant was detected by ELISA experiment. The collected cells from each group were washed three times with cold PBS. For every 1×10\hat{ }6 cells, 200 μL of lysis buffer was added to break the cells. The extract was centrifuged at 10,000 rpm for 10 minutes, and the supernatant was taken for detection.

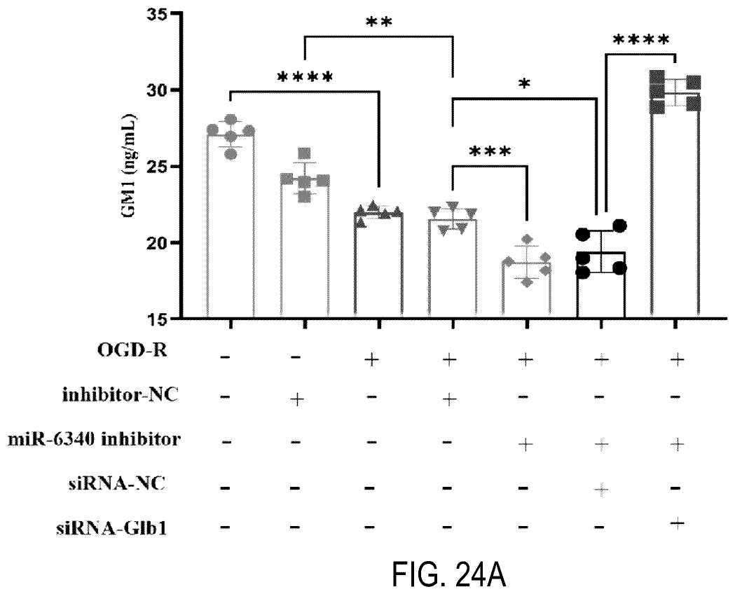

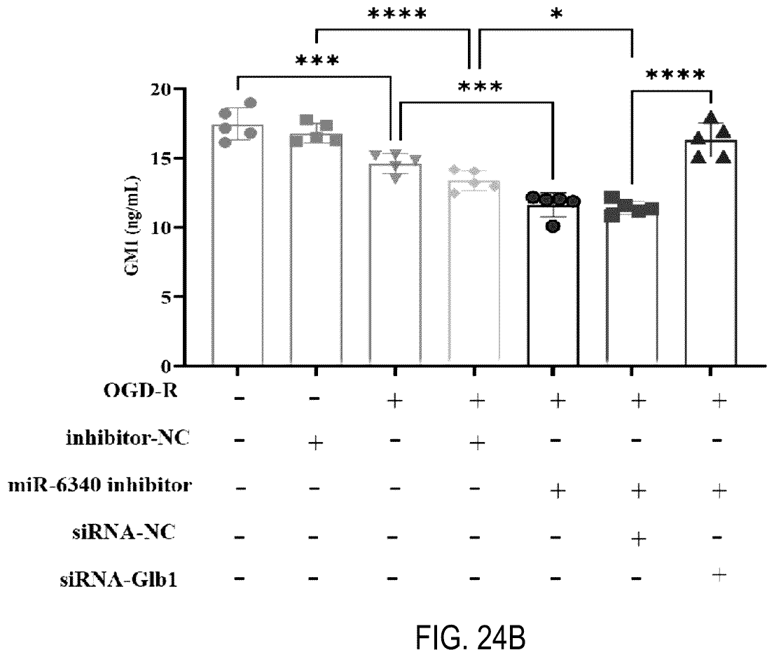

Both the cell culture supernatant samples and the cell lysate samples' ELISA results showed (FIGS. 24A and 24B), compared with the normal group, the expression of GM1 was downregulated in the OGD/R model group (P<0.0001); compared with the control group treated with inhibitor-NC, the expression of GM1 was downregulated in the microglia of the OGD/R model group treated with inhibitor-NC (P<0.01); compared with the OGD/R model group treated with inhibitor-NC, the expression of GM1 was downregulated in the microglia of the OGD/R model group treated with miR-6340 inhibitor (P<0.001); compared with the OGD/R model group treated with inhibitor-NC, the expression of GM1 was downregulated in the microglia of the OGD/R model group treated with both miR-6340 inhibitor and siRNA-NC (P<0.05); compared with the OGD/R model group treated with both miR-6340 inhibitor and siRNA-NC, the expression of GM1 was upregulated in the microglia of the OGD/R model group treated with both miR-6340 inhibitor and Glb1-siRNA (P<0.0001), indicating that OGD/R induces the synthesis of GM1 ganglioside in microglia through the miR-6340/Glb1 axis. In addition, the experimental results showed that, compared with the cell lysate samples, GM1 had a higher concentration in the cell supernatant and the data changes were more significant in each group. According to relevant literature, GM1 is an acidic glycosphingolipid that forms lipid rafts in the outer leaflet of the plasma membrane, especially in neurons of the central nervous system. They are involved in cell proliferation, differentiation, adhesion, signal transduction, cell-cell interactions, and tumorigenesis. Therefore, this application further confirms that the concentration of GM1 in BV2 cells decreases after OGD/R, and subsequently, the signal transduction and interactions between cells may also be reduced, which requires further experimental verification.

3.5 miR-6340/Glb1 Promotes Autophagy in BV2 Cells Through GM1 Metabolism
3.5.1 RT-qPCR was Used to Detect the mRNA Levels of Autophagy Markers in BV2 Cells after Downregulating Glb1 Expression.

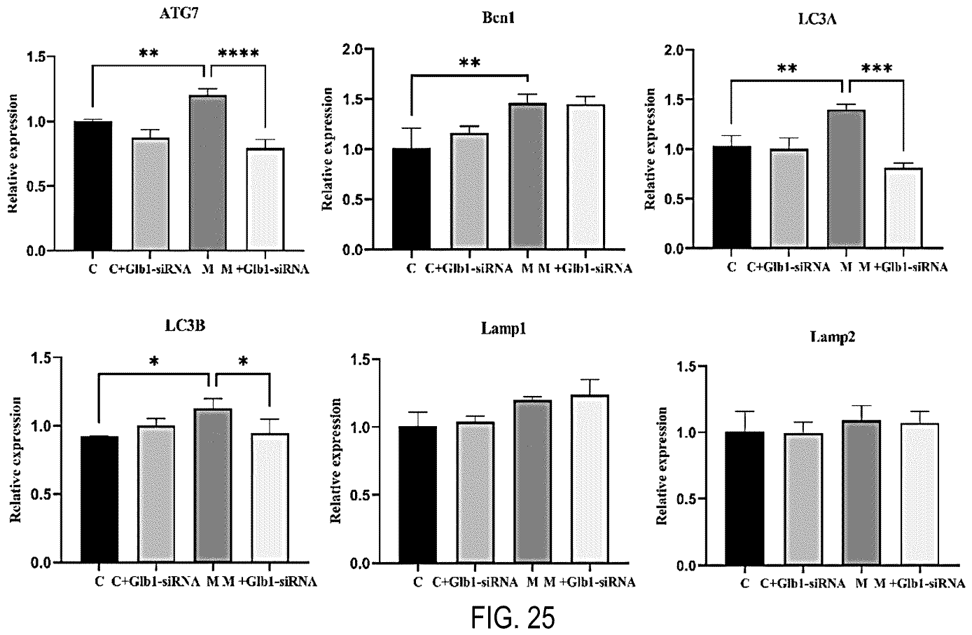

Existing technology has found that oxidative stress culture leads to an increase in aging-related β-galactosidase (SA-Glb1/SAB-gal), causing significant changes in the lysosomal system, including increased lysosomal mass and the content of autophagic vacuoles. Additionally, the GM1 metabolic pathway is closely related to cellular autophagy. Therefore, this application used RT-qPCR to detect the mRNA levels of autophagy markers in BV2 cells after downregulating Glb1 expression. The RT-qPCR results showed (FIG. 25), compared with the Control group, after OGD/R treatment in BV2 cells, the mRNA levels of ATG7, Bcn1, LC3A, and LC3B were all elevated and statistically significant, while the mRNA levels of Lamp1 and Lamp2 showed no significant difference but still had an increasing trend, thus indicating that cellular autophagy levels increased after OGD/R treatment. Compared with the Model, the mRNA levels of ATG7, LC3A, and LC3B in the Model+Glb1-siRNA group were significantly reduced and statistically significant, indicating that after suppressing Glb1 expression, the mRNA levels of autophagy markers in BV2 cells decreased, and cellular autophagy levels declined.

3.5.2 Transmission Electron Microscopy Observation of Autophagy in BV2 Cells after Transfection with Glb1-siRNA

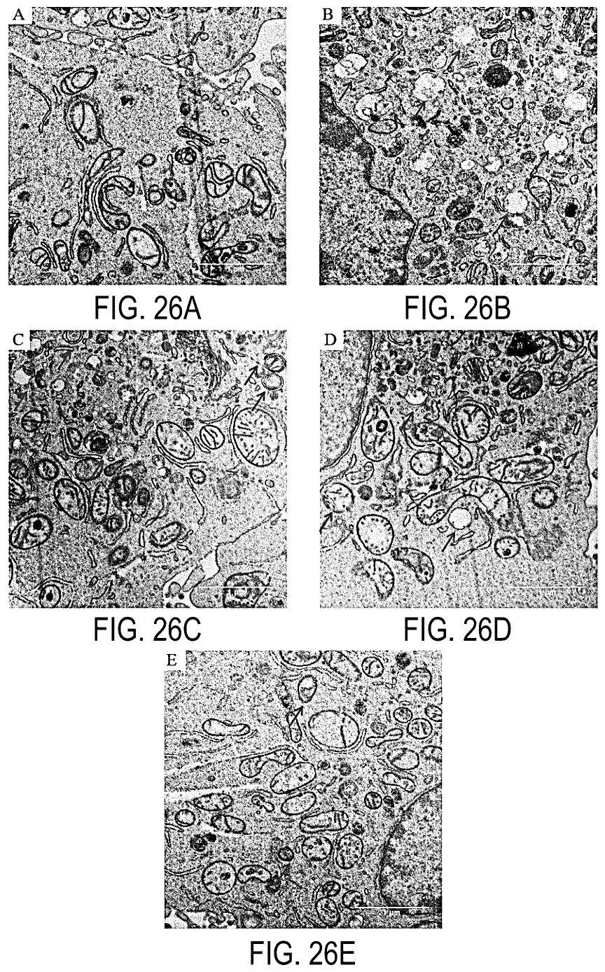

Transmission electron microscopy observations revealed that all groups of specimens were well observable under the microscope, with good staining and uniform coloring of the cell nuclei and cytoplasm, indicating that the fixation, sectioning, and specimen preparation met the standards. Further observation found that compared to the normal group cells (FIG. 26A), the cell nuclei in the OGD/R group specimens were significantly enlarged, and there were autophagic vesicle structures with double membranes (FIG. 26B). In the OGD/R+mir-6340 inhibitor group and OGD/R+siRNA-NC group, a large number of vesicles wrapped in single membranes were visible, and the cytoplasmic components had been degraded (FIGS. 26C and 26D). Compared with the OGD/R group, the OGD/R+Glb1-siRNA group occasionally showed autophagic bodies within the cells (FIG. 26E), and the degree of mitochondrial damage was reduced. Treatment with the OGD/R+mir-6340 inhibitor group led to increased expression of Glb1 within the cells, while treatment with the OGD/R+Glb1-siRNA group led to decreased expression of Glb1 within the cells. The experimental results indicated that after OGD/R, the expression of Glb1 within the cells increased, and the level of autophagy within the cells also increased. Therefore, we have reason to infer that after OGD/R treatment, miR-6340/Glb1 contributes to cell damage by participating in the autophagy process of BV2 cells.

What is claimed is:

1. Application of miR-6340 in the preparation of drug for treating cerebral ischemia.

2. The application according to claim 1, wherein the treatment is achieved by reducing a size of a cerebral infarction.

3. The application according to claim 2, wherein the reduction of the size of the cerebral infarction is achieved by down-regulating an expression of Glb1 to inhibit GM1 metabolism.

4. The application according to claim 1, wherein the treatment is achieved by reducing cell damage and autophagy.

5. The application according to claim 4, wherein the fact that the autophagy is a process in which BV2 cells is regulated by miR-6340/Glb1 through GM1 metabolism.

6. The application according to claim 4, wherein the reduction of the cell damage is achieved by down-regulating an expression of Glb1 to inhibit GM1 metabolism in microglia after OGD/R.

7. A drug for treating cerebral ischemia, comprising miR-6340.


SEQUENCE LISTING (39 sequences)

SEQ ID NO: 1 - DNA, 23 bp, synthetic construct
  source 1..23: mol_type = other DNA
aagttcaagacggtgacggaagc

SEQ ID NO: 2 - DNA, 25 bp, synthetic construct
  source 1..25: mol_type = other DNA
ccagttggtgaggacagtgaagttg

SEQ ID NO: 3 - DNA, 22 bp, synthetic construct
  source 1..22: mol_type = other DNA
gtccatgccatcactgccactc

SEQ ID NO: 4 - DNA, 22 bp, synthetic construct
  source 1..22: mol_type = other DNA
cgcctgcttcaccaccttcttg

SEQ ID NO: 5 - DNA, 23 bp, synthetic construct
  source 1..23: mol_type = other DNA
atgaagatggctgggctgaatgc

SEQ ID NO: 6 - DNA, 25 bp, synthetic construct
  source 1..25: mol_type = other DNA
caggaagcgtaggtagtcgtaatcg

SEQ ID NO: 7 - DNA, 23 bp, synthetic construct
  source 1..23: mol_type = other DNA
tcaccatcttccaggagcgagac

SEQ ID NO: 8 - DNA, 23 bp, synthetic construct
  source 1..23: mol_type = other DNA
tgagcccttccacaatgccaaag

SEQ ID NO: 9 - DNA, 25 bp, synthetic construct
  source 1..25: mol_type = other DNA
cccagaagaagttgaacgagtaccg

SEQ ID NO: 10 - DNA, 23 bp, synthetic construct
  source 1..23: mol_type = other DNA
gcagaccagcagagtcaccattg

SEQ ID NO: 11 - DNA, 24 bp, synthetic construct
  source 1..24: mol_type = other DNA
gagcgagttggtcaagatcatccg

SEQ ID NO: 12 - DNA, 24 bp, synthetic construct
  source 1..24: mol_type = other DNA
catagatgtcagcgatgggtgtgg

SEQ ID NO: 13 - DNA, 25 bp, synthetic construct
  source 1..25: mol_type = other DNA
caagccttcttcctcctggtgaatg

SEQ ID NO: 14 - DNA, 23 bp, synthetic construct
  source 1..23: mol_type = other DNA
ccattgctgtcccgaatgtctcc

SEQ ID NO: 15 - DNA, 21 bp, synthetic construct
  source 1..21: mol_type = other DNA
cgcaaggtggtggcagagaac

SEQ ID NO: 16 - DNA, 25 bp, synthetic construct
  source 1..25: mol_type = other DNA
actcactatactcccgctggtactg

SEQ ID NO: 17 - DNA, 24 bp, synthetic construct
  source 1..24: mol_type = other DNA
ggtttgggtctgtggaagagtgtg

SEQ ID NO: 18 - DNA, 24 bp, synthetic construct
  source 1..24: mol_type = other DNA
aggtaggcaatgaggacgatgagg

SEQ ID NO: 19 - DNA, 24 bp, synthetic construct
  source 1..24: mol_type = other DNA
ccaactccaactccaactccaacc

SEQ ID NO: 20 - DNA, 24 bp, synthetic construct
  source 1..24: mol_type = other DNA
ggcaccttctcctcagtgatgttc

SEQ ID NO: 21 - DNA, 23 bp, synthetic construct
  source 1..23: mol_type = other DNA
tcctcggcctctgattcctcttg

SEQ ID NO: 22 - DNA, 23 bp, synthetic construct
  source 1..23: mol_type = other DNA
cttacaccacggcacgaactctc

SEQ ID NO: 23 - DNA, 21 bp, synthetic construct
  source 1..21: mol_type = other DNA
gcacaggcaactaccgcatcc

SEQ ID NO: 24 - DNA, 24 bp, synthetic construct
  source 1..24: mol_type = other DNA
cccttgagagaggcagcaatcttg

SEQ ID NO: 25 - DNA, 25 bp, synthetic construct
  source 1..25: mol_type = other DNA
gtcactcacatctacacagcacagg

SEQ ID NO: 26 - DNA, 23 bp, synthetic construct
  source 1..23: mol_type = other DNA
aattctgccagcacacggatacc

SEQ ID NO: 27 - DNA, 24 bp, synthetic construct
  source 1..24: mol_type = other DNA
cttcaggacatgcggacgttacag

SEQ ID NO: 28 - DNA, 25 bp, synthetic construct
  source 1..25: mol_type = other DNA
ataggatcgtgccgttcaacatacc

SEQ ID NO: 29 - DNA, 25 bp, synthetic construct
  source 1..25: mol_type = other DNA
gaggacaggtacagcatcaacttgg

SEQ ID NO: 30 - DNA, 24 bp, synthetic construct
  source 1..24: mol_type = other DNA
aagtcatgttcgggcagcatatcc

SEQ ID NO: 31 - DNA, 21 bp, synthetic construct
  source 1..21: mol_type = other DNA
cgcagctatgacgcctgtgac

SEQ ID NO: 32 - DNA, 22 bp, synthetic construct
  source 1..22: mol_type = other DNA
agggtgggctggattggagaag

SEQ ID NO: 33 - DNA, 21 bp, synthetic construct
  source 1..21: mol_type = other DNA
aaggggtcgctcaagggtcag

SEQ ID NO: 34 - DNA, 24 bp, synthetic construct
  source 1..24: mol_type = other DNA
gctgtaggttcattctccgctgtc

SEQ ID NO: 35 - DNA, 21 bp, synthetic construct
  source 1..21: mol_type = other DNA
acaacaacgtcagcagcagct

SEQ ID NO: 36 - DNA, 18 bp, synthetic construct
  source 1..18: mol_type = other DNA
cagtgcgtgtcgtggagt

SEQ ID NO: 37 - DNA, 50 bp, synthetic construct
  source 1..50: mol_type = other DNA
gtcgtatccagtgcagggtccgaggtattcgcactggatacgacgccaaa

SEQ ID NO: 38 - DNA, 20 bp, synthetic construct
  source 1..20: mol_type = other DNA
atccagtgcagggtccgagg

SEQ ID NO: 39 - DNA, 20 bp, synthetic construct
  source 1..20: mol_type = other DNA
aacgcttcacgaatttgcgt